(19) United States
(12) Patent Application Publication
(10) Pub. No.: US 2016/0271031 A1
(43) Pub. Date: Sep. 22, 2016

(54) PRODUCT FOR HAIR COLOURING WITH INTENSIVE CONDITIONING

(71) Applicant: Henkel AG & Co. KGaA, Duesseldorf (DE)

(72) Inventors: Matthias Schweinsberg, Hamburg (DE); Torsten Lechner, Hamburg (DE)

(73) Assignee: Henkel AG & Co. KGaA, Duesseldorf (DE)

(21) Appl. No.: 15/168,401

(22) Filed: May 31, 2016

Related U.S. Application Data
(63) Continuation of application No. PCT/EP2014/074848, filed on Nov. 18, 2014.

(30) Foreign Application Priority Data
Dec. 16, 2013 (DE) 10 2013 226 074.1

(51) Int. Cl. A61K 8/41; A61K 8/81; A61K 8/60; A61Q 5/10; A61Q 5/12

(52) U.S. Cl. CPC A61K 8/416; A61Q 5/10; A61Q 5/12; A61K 8/60; A61K 8/8176; A61K 8/817; A61K 2800/884; A61K 2800/4324

(57) ABSTRACT

The present invention relates to a product for coloring and/or lightening keratinic fibers, more particularly human hair, and to a method for employing this product. The product of the invention comprises three compositions, which are kept separate from one another until being employed. The invention further relates to the use of this product for improving absorption of color in the coloring of keratinic fibers, the use thereof to enhance the wash stability of colored keratinic fibers, and the use thereof to enhance the conditioning of keratinic fibers.

### FIELD OF THE INVENTION

The present invention generally relates to an agent for coloring and/or lightening keratinic fibers, in particular human hair, and to a method for using said agent. The agent of the invention comprises three compositions kept separated until use. The invention furthermore relates to the use of this agent for improving color absorption during the dyeing of keratinic fibers, to the use thereof to improve the wash fastness of dyed keratinic fibers, and to the use thereof to improve the conditioning of keratinic fibers.

### BACKGROUND OF THE INVENTION

Human hair is treated in many ways today with hair cosmetic preparations. These include, for instance, cleansing the hair with shampoos, care and regeneration with rinses and treatments, and bleaching, coloring, and shaping the hair using dyes, tints, waving compositions, and styling preparations. In this connection, agents for modifying or nuancing the color of head hair play a prominent role.

Apart from bleaching agents, which bring about an oxidative lightening of the hair by breaking down the natural hair dyes, essentially three types of hair dyes are of importance in field of hair dyeing: So-called oxidation dyes are used for long-lasting, intensive colors with suitable fastness properties. Such dyes customarily include oxidation dye precursors, so-called developer components and coupler components. Under the influence of oxidizing agents or atmospheric oxygen, the developer components form the actual dyes with one another or during coupling with one or more coupler components. The oxidation dyes are characterized by excellent, long-lasting color results. For natural-looking colors, however, customarily a mixture of a relatively large number of oxidation dye precursors must be used; in many cases, direct dyes are used, furthermore, for providing nuances.

Coloring or tinting agents, including so-called direct dyes as the coloring component, are customarily used for temporary colors. These are dye molecules that are directly absorbed onto the hair and do not require any oxidative process to develop the color. These dyes include, for example, henna which was already known in antiquity for dyeing skin and hair. These dyes are as a rule markedly more sensitive to shampooing than the oxidative dyes, so that then an often undesirable shift in nuances or even a visible “decolorizing” occurs much more rapidly.

Lastly, a novel dyeing method has attracted great interest recently. In this method, precursors of the natural hair dye melanin are applied to the hair; these then form bioanalogous dyes in the course of oxidative processes in the hair. 5,6-Dihydroxyindoline, for example, is used as the dye precursor in such a method. In the case of particularly repeated use of agents including 5,6-dihydroxyindoline, it is possible to restore the natural hair color to humans having gray hair. Coloring can occur here with atmospheric oxygen as the sole oxidizing agent, so that no other oxidizing agents need to be utilized. In persons originally having medium-blond to brown hair, indoline can be used as the sole dye precursor. For use in persons having an originally red and in particular dark to black hair color, in contrast, satisfactory results can often be obtained only by the concurrent use of further dye components, in particular special oxidation dye precursors.

After the coloring process, hair is exposed to a wide variety of environmental stresses over a long period of time. These extend from everyday weathering of the hair, for example, by sunlight and hair washing, to mechanical stresses brought about by styling, to chemical influences if the consumer subjects the hair to a subsequent hair coloring or shaping process. Environmental influences have considerable effects not only on the hair structure itself but also on the dyes present in the hair after the coloring process. The dyes may be bleached out by light exposure, or may be washed out of the hair by perspiration or shampooing. These properties are referred to as the fastness properties of the dyes. If the dyes formed or directly used in the course of color formation have appreciably different fastness properties (e.g., UV stability, perspiration resistance, wash fastness, etc.), a detectable and therefore undesired color shift may then occur over time. Both the occurrence of color shifts and, in particular, the fading of the colors caused by washing are not desired by the consumer.

There are various possibilities for reducing the loss of hair color intensity caused by repeated washing. The intensity decrease perceived by the consumer may be delayed by striving for a color result that is as intense as possible from the outset. A second possibility is to improve color retention by using active substances that inhibit or reduce the process by which dyes are washed out. It is particularly advantageous to discover an active substance or active substance combination that both improves the color absorption capability of the dyes during the coloring process and minimizes the later washing out of the dyes upon subsequent shampooing. To implement the second possibility, both pretreatment with compositions including special active substances, which inhibit or reduce the dye washing out process, and also an after-treatment with compositions including special active substances, which inhibit or reduce the dye washout process, and lastly the addition of these special active substances directly to the dye cream are used in the state of the art. Improving the color absorption and inhibiting the dye washout process and improving the conditioning of the keratinic fibers do not succeed in any event with these compositions according to the state of the art.

The object of the present invention, therefore, is to provide agents for coloring and/or lightening keratinic fibers, which improve color absorption during dyeing and intensify the penetration of the dyes or dye precursors into the keratinic fibers and provide excellent conditioning, so that ideally subsequent treatment with conditioning compositions such as rinses or treatments can be omitted. Furthermore, washing out of dyes during repeated shampooing is to be prevented or minimized. The agents of the invention are to improve the color intensity and wash fastness, without having the disadvantages intrinsic to the dyes already known from the state of the art.

Moreover, colors are to be achieved with these agents which have an advantageous performance profile in regard to their other fastness properties, such as, for example, light resistance, rubbing fastness, perspiration resistance, and cold waving fastness. Lastly, it is especially desirable to provide dyes with good levelling properties.

In the cosmetics market, optimizing color retention is very important, and there are already different color-protecting shampoos or conditioners on the market. Various substances used for color protection of previously colored hair are known from the state of the art.

“Silicones Used in Permanent and Semi-Permanent Hair Dyes to Reduce the Fading and Color Change Process of Dyed Hair Occurring by Wash-Out or UV Radiation”, J. Cosmetic Sci., (2004) 55 (Supplement), pp. 123-131, describes the use of silicones for improving color retention.

U.S. Pat. No. 7,066,966 and U.S. Pat. No. 7,147,672 disclose an oxidative dye composition with good wash fastness including a cationic poly(vinyllactam).

US 2011/0219552 A1 discloses a method for protecting dyed hair from wash-out by using hydrophobized cationic polymers.

Lastly, US 2011/0044924 relates to agents and methods for enhancing color intensity and for color protection of dyed hair, which include quaternary ammonium salts.

It was found completely surprisingly and unexpectedly that an agent including a dyeing or bleaching composition including dye precursors, a developer composition including oxidizing agents, and furthermore a composition including a conditioning composition is characterized in that all three compositions, conventional per se, are mixed together immediately before use and applied to keratinic fibers, which results in a marked reduction of the color shift and in excellent conditioning of the keratinic fibers dyed with said agents.

Furthermore, other desirable features and characteristics of the present invention will become apparent from the subsequent detailed description of the invention and the appended claims, taken in conjunction with this background of the invention.

### BRIEF SUMMARY OF THE INVENTION

An agent for coloring and/or lightening keratinic fibers, in particular human hair, including in each case in a cosmetic carrier a composition including dye precursors, a composition including an oxidizing agent, and a conditioning composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present invention comprises agents for coloring and/or lightening keratinic fibers, in particular human hair, including in each case in a cosmetic carrier
(a) a composition including dye precursors,
(b) a composition including an oxidizing agent, and
(c) a conditioning composition.

To prevent a premature, undesirable reaction of the dye precursors with the oxidizing agent, dye precursors and oxidizing agents are expediently produced separately from one another in compositions (a) and (b) and are brought into contact only immediately before use. At this time, composition (c) of the invention is brought into contact with compositions (a) and (b).

Keratin-including fibers are understood in principle to be all animal hair, e.g., for example, wool, horsehair, angora hair, fur, feathers, and products or textiles manufactured therefrom. Preferably, however, the keratinic fibers are human hair.

The phrase used according to the invention “coloring of keratin fibers” comprises any form of color modification of the fibers. In particular, color modifications falling within the terms tinting, lightening, blonding, bleaching, oxidative coloring, semipermanent coloring, permanent coloring, and temporary coloring are included. Color modifications according to the invention which have a lighter coloring result than the initial color, such as, for example, blonding with coloring, are explicitly also included.

The composition (a) of the invention is described below.

Compositions (a) of the invention include at least one color-modifying compound as the essential ingredient. A color-modifying compound in the context of the present invention is understood to be a compound or a substance by means of which the color of the keratinic fibers can be modified. This definition includes both compounds that modify keratinic fibers in the direction of a darker shade and compounds after the use of which the keratinic fibers have a lighter shade than the original shade. The definition of the color-modifying compound includes oxidation dye precursors, direct dyes, dye precursors of bioanalogous dyes, and oxidizing agents.

In a further preferred embodiment, composition (a) includes at least one oxidation dye precursor and/or at least one direct dye for coloring and/or lightening keratinic fibers, therefore as a color-modifying compound.

Oxidation dye precursors include oxidation dye precursors of the developer type and of the coupler type. Especially suitable oxidation dye precursors of the developer type are thereby selected from at least one compound from the group formed by p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4, 5-diamino-1-(2-hydroxyethyl)pyrazole, 2,4,5, 6-tetraaminopyrimidine, 4-hydroxy-2, 5,6-triaminopyrimidine, 2-hydroxy-4,5,6-triaminopyrimidine, 2,3-diamino-6,7-dihydro-1H,5H-pyrazolo[1,2-a]pyrazol-1-one, and the physiologically acceptable salts thereof.

Especially suitable oxidation dye precursors of the coupler type are selected from the group formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4, 5-dimethylphenyl}amino) ethanol, 2-[3-morpholin-4-ylphenyl)amino]ethanol, 3-amino-4-(2-methoxyethoxy)-5-methyl phenyl amine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3, 4-dimethylpyridine, 3,5-diamino-2,6-dimethoxypyridine, 1-phenyl-3-methylpyrazol-5-one, 1-naphthol, 1,5-dihydroxynaphthalene, 2, 7-dihydroxynaphthalene, 1, 7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

It has been found in the context of work leading to this invention that the object of the invention is achieved to a particular degree using compositions (a) including certain developer/coupler combinations. Therefore, compositions (a) for coloring keratinic fibers that include specific combinations of oxidation dye precursors are particularly preferred.

It is preferred, if the compositions (a) of the invention include of the following developer/coupler combinations.
p-toluylenediamine/resorcinol
p-toluylenediamine/2-methylresorcinol
p-toluylenediamine/2-amino-3-hydroxypyridine
p-toluylenediamine/2,7-dihydroxynaphthalene
p-toluylenediamine/3-aminophenol
p-toluylenediamine/1-naphthol
p-toluylenediamine/1,5-dihydroxynaphthalene
p-toluylenediamine/5-amino-2-methylphenol
p-toluylenediamine/2-(2,4-diaminophenoxy)ethanol
p-toluylenediamine/3-amino-2-chloro-6-methylphenol
p-toluylenediamine/2,6-dihydroxy-3,4-dimethylpyridine
p-toluylenediamine/3-amino-2-methylamino-6-methoxypyridine
p-toluylenediamine/1,3-bis(2,4-diaminophenyl)propane
2,4,5,6-tetraaminopyrimidine/resorcinol
2,4,5,6-tetraaminopyrimidine/2-methylresorcinol
2,4,5,6-tetraaminopyrimidine/2-amino-3-hydroxypyridine
2,4,5,6-tetraaminopyrimidine/2,7-dihydroxynaphthalene
4,5-diamino-1-(2-hydroxyethyl)pyrazole/resorcinol
4,5-diamino-1-(2-hydroxyethyl)pyrazole/2-methylresorcinol
4,5-diamino-1-(2-hydroxyethyl)pyrazole/2-amino-3-hydroxypyridine
4,5-diamino-1-(2-hydroxyethyl)pyrazole/2,7-dihydroxynaphthalene
p-aminophenol/resorcinol
p-aminophenol/2-methylresorcinol
p-aminophenol/2-amino-3-hydroxypyridine
p-aminophenol/2,7-dihydroxynaphthalene
p-aminophenol/3-aminophenol
p-aminophenol/1-naphthol
p-aminophenol/1,5-dihydroxynaphthalene
p-aminophenol/5-amino-2-methylphenol
p-aminophenol/2-(2,4-diaminophenoxy)ethanol
p-aminophenol/3-amino-2-chloro-6-methylphenol
p-aminophenol/2,6-dihydroxy-3,4-dimethylpyridine
p-aminophenol/3-amino-2-methylamino-6-methoxypyridine
p-aminophenol/1,3-bis(2,4-diaminophenyl)propane.

Particularly preferred compositions (a) of the invention include one of the following combinations of two developers and one coupler:
p-toluylenediamine/2,4,5,6-tetraaminopyrimidine/resorcinol
p-toluylenediamine/2,4,5,6-tetraaminopyrimidine/2-methylresorcinol
p-toluylenediamine/2,4,5,6-tetraaminopyrimidine/2-amino-3-hydroxypyridine
p-toluylenediamine/2,4,5,6-tetraaminopyrimidine/2,7-dihydroxynaphthalene.

An especially good color intensification or especially good color retention is achieved in particular when the keratinic fibers are colored with a formulation including the combination: p-toluylenediamine, 2,4,5,6-tetraaminopyrimidine, resorcinol, 2-methylresorcinol, 2-amino-3-hydroxypridine, and 2,7-dihydroxynaphthalene.

In this case, the developer components and coupler components are generally used in approximately molar amounts to one another. Although molar use has proven to be expedient, a certain excess of individual oxidation dye precursors is not disadvantageous, so that developer components and coupler components may have a molar ratio of 1:0.5 to 1:3, particularly 1:1 to 1:2.

In the context of a further preferred embodiment, composition (a) of the invention includes as the color-modifying compound at least one direct dye. These are dyes that are directly absorbed onto the hair and do not require any oxidative process to develop the color. Direct dyes are usually nitrophenylenediamines, nitroaminophenols, azo dyes, anthraquinones, or indophenols.

Direct dyes can be divided into anionic, cationic, and nonionic direct dyes, which are selected and used by the skilled artisan in accordance with the requirements of the carrier base.

Preferred anionic direct dyes are the compounds known under the international names or trade names: bromophenol blue, tetrabromophenol blue, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, and Acid Black 52.

Preferred cationic direct dyes are Basic Blue 7, Basic Blue 26, Basic Violet 2 and Basic Violet 14, Basic Yellow 57, Basic Red 76, Basic Blue 16, Basic Blue 347 (Cationic Blue 347/Dystar), Basic Blue 99, Basic Brown 16 and Basic Brown 17, as well as Yellow 87, Basic Orange 31, and Basic Red 51.

Nonionic nitro and quinone dyes, and neutral azo dyes, are particularly suitable as nonionic direct dyes. Preferred nonionic direct dyes are compounds known under the international names or trade names: HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, and Disperse Black 9, as well as 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

It is preferred if composition (a) of the invention includes as a color-modifying compound the direct dye 4-amino-3-nitrophenol either alone or in combination with other color-modifying compounds.

It is not required that the optionally present direct dyes each represent pure compounds. Rather, due to the production methods for the individual dyes, minor amounts of other components can be present, provided these have no detrimental effect on the coloring result or should be excluded for other reasons, e.g., toxicological.

In a further preferred embodiment, compositions (a) of the invention include as color-modifying compounds both a direct dye and also an oxidation dye precursor of developer and/or coupler type.

Composition (a) of the invention can also include as the color-modifying compound further at least one dye precursor of a bioanalogous dye. The dye precursors of bioanalogous dyes that are used are preferably indoles and indolines which have at least two groups selected from hydroxy and/or amino groups, preferably as substituents on the six-membered ring. These groups can carry further substituents, e.g., in the form of an etherification or esterification of the hydroxy group or alkylation of the amino group.

Compositions of the invention that include the precursors of bioanalogous dyes are used preferably as air-oxidative dyes. In this embodiment, said compositions consequently are not combined with an additional oxidizing agent.

Particularly preferred indoline derivatives are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, and 5,6-dihydroxyindoline-2-carboxylic acid.

Particularly preferred indole derivatives are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, and 5,6-dihydroxyindole-2-carboxylic acid.

Composition (a) of the invention includes at least one color-modifying compound in each case in a weight proportion of 0.001 to 12%. If the color-modifying composition (a) constitutes oxidation dye precursors, direct dyes, and/or precursors of bioanalogous dyes, they are preferably each employed in an amount of 0.01 to 10% by weight, particularly preferably of 0.1 to 5% by weight, and especially preferably of 0.25 to 3% by weight, based on the ready-to-use agent.

A further particularly preferred embodiment of the first subject of the invention is therefore a composition (a) for coloring and/or lightening keratinic fibers, which is characterized in that it includes as the color-modifying compound at least one oxidation dye precursor and/or at least one direct dye, in each case in an amount of 0.001 to 12% by weight, preferably of 0.01 to 10% by weight, particularly preferably of 0.1 to 5% by weight, and especially preferably of 0.25 to 3% by weight, based on the weight of the combined compositions (a) and (b).

The agents of the invention can be used as lightening dyes or as lighteners. In order to achieve the lightening effect, compositions (b) include for this purpose an oxidizing agent of the color-modifying compound. Hydrogen peroxide and/or a solid adduct thereof to organic or inorganic compounds are used preferably as the oxidizing agent. Examples of such adducts are adducts to urea, melamine, and sodium borate.

In a preferred embodiment, hydrogen peroxide itself is used as an aqueous solution. The concentration of a hydrogen peroxide solution in the agent of the invention is determined, on the one hand, by legal requirements and, on the other, by the desired effect; preferably, 6 to 12% by weight solutions in water are used.

Ready-to-use agents of the first subject of the invention that are preferred according to the invention are characterized in that they include the oxidizing agent in an amount of 0.001 to 12% by weight, preferably of 0.01 to 10% by weight, preferably 1 to 9% by weight, particularly preferably 2.5 to 8% by weight, and in particular 3 to 6% by weight, based on the weight of the combined compositions (a) and (b).

A further particularly preferred embodiment of composition (b), therefore, is an agent for coloring and/or lightening keratinic fibers, which characterized in that it includes as the color-modifying compound at least one oxidizing agent, selected from hydrogen peroxide and the solid adducts thereof to organic or inorganic compounds, in an amount of 0.001 to 12% by weight, preferably of 0.01 to 10% by weight, primarily 1 to 9% by weight, particularly preferably 2.5 to 8% by weight, and in particular 3 to 6% by weight, based on the weight of the combined compositions (a) and (b).

Such oxidizing agent preparations are preferably aqueous, flowable oxidizing agent preparations. In this case, preferred preparations are characterized in that the flowable oxidizing agent preparation, based on its weight, includes 40 to 90% by weight, preferably 50 to 85% by weight, particularly preferably 55 to 80% by weight, more preferably 60 to 77.5% by weight, and in particular 65 to 75% by weight of water.

In the case of oxidative dyeing, the development of the color can basically occur with atmospheric oxygen. A chemical oxidizing agent is preferably used, however especially when apart from coloring a lightening effect on human hair is desired. This lightening effect can be desirable independent of the coloring method. Hydrogen peroxide in particular and/or one of its stable adducts to organic or inorganic compounds may be suitable as oxidizing agents.

In a further preferred embodiment, the agents of the invention therefore include as color-modifying compounds both an oxidation dye precursor of the developer and/or coupler type, as well as an oxidizing agent.

To achieve an intensified lightening and bleaching effect, the agent can include furthermore at least one peroxo salt. Suitable peroxo salts are inorganic peroxo compounds, preferably selected from the group formed by ammonium peroxodisulfate, alkali metal peroxodisulfates, ammonium peroxomonosulfate, alkali metal peroxomonosulfates, alkali metal peroxodiphosphates, and alkaline earth metal peroxides. Peroxodisulfates, in particular ammonium peroxodisulfate, potassium peroxodisulfate, and sodium peroxodisulfate are particularly preferred.

A further preferred embodiment of the first subject of the invention, therefore, is a composition (b) of the invention, which is characterized in that it includes in addition, based on its weight, 0.01 to 30% of a bleach booster, selected from ammonium peroxodisulfate, potassium peroxodisulfate, sodium peroxodisulfate, potassium hydrogen peroxomonosulfate, potassium peroxodiphosphate, magnesium peroxide, and barium peroxide.

If compositions (b) additionally include persulfates, composition (b) includes these persulfates up to 0.01 to 30% by weight, preferably to 1.5 to 28% by weight, primarily 2.0 to 25% by weight, and in particular up to 5 to 20% by weight, based in each case on the weight of the combined compositions (a) and (b).

In order to intensify the bleaching effect, composition (b) can include further bleach boosters such as, for example, tetraacetylethylenediamine (TAED), 1,5-diacetyl-2,4-dioxohexahydro-1,3,5-triazine (DADHT), tetraacetyl glycoluril (TAGU), N-nonanoylsuccinimide (NOSI), n-nonanoyl- or isononanoyloxybenzenesulfonate (n- or i-NOBS), phthalic acid anhydride, triacetin, ethylene glycol diacetate, and 2,5-diacetoxy-2,5-dihydrofuran, and carbonate salts or hydrogen carbonate salts, in particular ammonium hydrogen carbonate, ammonium carbonate, sodium hydrogen carbonate, disodium carbonate, potassium hydrogen carbonate, dipotassium carbonate, and calcium carbonate, and nitrogen-containing, heterocyclic bleach boosters, such as 4-acetyl-1-methylpyridinium-p-toluenesulfonate, 2-acetyl-1-methylpyridinium-p-toluenesulfonate, and N-methyl-3,4-dihydroisoquinolinium-p-toluenesulfonate.

To further enhance the lightening, at least one SiO₂ compound, such as silicic acid or silicates, in particular water glasses, can be added in addition to the composition of the invention. It can be preferred according to the invention to use the SiO₂ compounds in amounts of 0.05% by weight to 15% by weight, particularly preferably in amounts of 0.15% by weight to 10% by weight, and very particularly preferably in amounts of 0.2% by weight to 5% by weight, based in each case on the anhydrous composition of the invention. The quantitative data in this case indicate the content of the SiO₂ compounds (without their water component) in the agents.

It has proven advantageous, furthermore, if the oxidizing agent preparations include at least one stabilizer or complexing agent. Common complexing agents and stabilizers that are preferred in the context of the present invention are, for example, polyoxycarboxylic acids, polyamines, ethylenediaminetetraacetic acid (EDTA), N-hydroxyethylethylenediaminetriacetic acid, diethylenetriaminepentaacetic acid (DTPA), ethylenediaminedisuccinic acid (EDDS), hydroxyethyliminodiacetic acid, nitridodiacetic acid-3-propionic acid, isoserinediacetic acid, N,N-di-(2-hydroxy-ethyl)glycine, N-(1,2-dicarboxy-2-hydroxyethyl)glycine, N-(1,2-dicarboxy-2-hydroxy-ethyl)aspartic acid or nitrilotriacetic acid (NTA), ethylenediaminediglutaric acid (EDGA), 2-hydroxypropylenediaminedisuccinic acid (HPDS), glycinamide-N,N′-disuccinic acid (GADS), ethylendiamine-N—N′-diglutaric acid (EDDG), 2-hydroxypropylenediamine-N—N′-disuccinic acid (HPDDS), diaminoalkyldi(sulfosuccinic acid) (DDS), ethylenedicysteic acid (EDC), ethylenediamine-N—N′-bis(ortho-hydroxyphenyl)acetic acid (EDDHA), N-2-hydroxyethylamine-N,N-diacetic acid, glyceryliminodiacetic acid, iminodiacetic acid-N-2-hydroxypropylsulfonic acid, aspartic acid-N-carboxymethyl-N-2, 5-hydroxypropyl-3-sulfonic acid, β-alanine-N,N′-diacetic acid, aspartic acid-N,N′-diacetic acid, aspartic acid-N-monoacetic acid, dipicolinic acid, as well as salts and/or derivatives thereof; geminal diphosphonic acids such as 1-hydroxyethane-1,1-diphosphonic acid (HEDP), higher homologs thereof having up to 8 carbon atoms, and hydroxy or amino group-containing derivatives thereof, and 1-aminoethane-1,1-diphosphonic acid, higher homologs thereof having up to 8 carbon atoms, and hydroxy or amino group-containing derivatives, aminophosphonic acids such as ethylenediaminetetra(methylenephosphonic acid) (EDTMP), diethylenetriaminepenta(methylenephosphonic acid) (DTPMP) and higher homologs thereof, or nitrilotri(methylenephosphonic acid), phosphonopolycarboxylic acids such as 2-phosphonobutane-1,2,4-tricarboxylic acid, cyclodextrins, and alkali stannates (sodium stannate), alkali pyrophosphates (tetrasodium pyrophosphate, disodium pyrophosphate), alkali phosphates (sodium phosphate), and phosphoric acid, as well as salts thereof.

To prevent a premature, undesirable reaction of the oxidation dye precursors with the oxidizing agent, oxidation dye precursors and oxidizing agents are expediently produced separately from one another and brought into contact only immediately before use.

Composition (c) of the invention is a conventional keratinic fiber-conditioning composition. Preferred compositions (c) of the invention are characterized in that at least one quaternary ammonium compound is present in a total amount of 0.1 to 10.0% by weight, whereby the quaternary ammonium compound is selected from at least one of the groups
i. the esterquats, and/or
ii. the quaternary imidazolines of the formula (Tkat2),

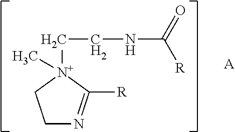

where the R group independently of one another stands in each case for a saturated or unsaturated, linear or branched hydrocarbon group with a chain length of 8 to 30 carbon atoms and A for a physiologically acceptable anion, and/or
iii. the amines and/or cationized amines, and/or
iv. poly(methacryloyloxyethyl)trimethylammonium compounds, and/or
v. quaternized cellulose derivatives, in particular Polyquaternium-10, Polyquaternium-24, Polyquaternium-27, Polyquaternium-67, Polyquaternium-72, and/or
vi. cationic alkyl polyglycosides, and/or
vii. cationized honey, and/or
viii. cationic guar derivatives, and/or
ix. chitosan, and/or
x. polymeric dimethyldiallylammonium salts and copolymers thereof with esters and amides of acrylic acid and methacrylic acid, in particular Polyquaternium-7, and/or
xi. copolymers of vinylpyrrolidone with quaternized derivatives of dialkylaminoalkyl acrylate and methacrylate, in particular Polyquaternium-11, and/or
xii. vinylpyrrolidone/vinylimidazolium methochloride copolymers, in particular Polyquaternium-16, and/or
xiii. quaternized polyvinyl alcohol, and/or
xiv. Polyquaternium-74, and
   mixtures thereof.

Cationic surfactants of the formula (Tkat1-1) form the first group of cationic surfactants.

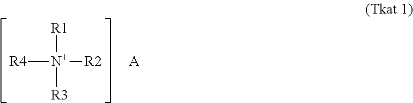

In the formula (Tkat1), R1, R2, R3, and R4 in each case independently of one another stand for hydrogen, a methyl group, a phenyl group, a benzyl group, for a saturated, branched or unbranched alkyl group having a chain length of 8 to 30 carbon atoms, which optionally can be substituted with one or more hydroxy groups. ‘A’ stands for a physiologically acceptable anion, for example, halides such as chloride or bromide and methosulfates.

Examples of compounds of the formula (Tkat1) are lauryl trimethyl ammonium chloride, cetyl trimethyl ammonium chloride, cetyl trimethyl ammonium bromide, cetyl trimethyl ammonium methosulfate, dicetyl dimethyl ammonium chloride, tricetyl methyl ammonium chloride, stearyl trimethyl ammonium chloride, distearyl dimethyl ammonium chloride, lauryl dimethyl benzyl ammonium chloride, behenyl trimethyl ammonium chloride, behenyl trimethyl ammonium bromide, and behenyl trimethyl ammonium methosulfate.

Esterquats according to the formula (Tkat2) form a preferred group.

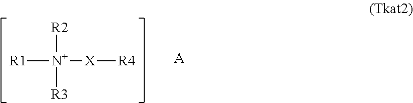

Herein, the groups R1, R2, and R3 are each independent of one another and may be the same or different. The groups R1, R2, and R3 denote:
a branched or unbranched alkyl group having 1 to 4 carbon atoms which can include at least one hydroxyl group, or
a saturated or unsaturated, branched or unbranched or a cyclic saturated or unsaturated alkyl group having 6 to 30 carbon atoms which can include at least one hydroxyl group, or
an aryl or alkaryl group, for example, phenyl or benzene,
the group (—X—R4), with the proviso that at most 2 of the groups R1, R2, or R3 can stand for this group:
   The group —(X—R4) is contained at least 1 to 3 times.

Herein, X stands for:
1) —(CH2)n- with n=1 to 20, preferably n=1 to 10, and particularly preferably n=1 to 5, or
2) —(CH2-CHR5-O)n- with n=1 to 200, preferably 1 to 100, particularly preferably 1 to 50, and especially preferably 1 to 20 with R5 having the meaning of hydrogen, methyl, or ethyl,
3) a hydroxy alkyl group having one to four carbon atoms, which can be branched or unbranched and which includes at least one and that most 3 hydroxy groups. Examples are: —CH₂OH, —CH₂CH₂OH, —CHOHCHOH, —CH₂CHOHCH₃, —CH(CH₂OH)₂, —C OH(CH₂OH)₂, —CH₂CHOHCH₂OH, —CH₂CH₂CH₂OH, and hydroxybutyl groups,
   and R4 stands for:
1) R6-O—CO—, where R6 is a saturated or unsaturated, branched or unbranched or a cyclic saturated or unsaturated alkyl group having 6 to 30 carbon atoms, which can include at least one hydroxy group, and which optionally can be oxethylated furthermore with 1 to 100 ethylene oxide units and/or 1 to 100 propylene oxide units, or
2) R7-CO—, where R7 is a saturated or unsaturated, branched or unbranched or a cyclic saturated or unsaturated alkyl group having 6 to 30 carbon atoms, which can include at least one hydroxy group, and which optionally can be oxethylated furthermore with 1 to 100 ethylene oxide units and/or 1 to 100 propylene oxide units,
   and ‘A’ stands for a physiologically acceptable organic or inorganic anion and is defined here to also represent all structures described below. The anion of all described cationic compounds is selected from the halide ions, fluoride, chloride, bromide, iodide, sulfates of the general formula RSO3⁻, where R has the meaning of saturated or unsaturated alkyl groups having 1 to 4 carbon atoms, or anionic groups of organic acids such as maleate, fumarate, oxalate, tartrate, citrate, lactate, or acetate.

Such products are marketed, for example, under the trademarks Rewoquat®, Stepantex®, Dehyquart®, Armocare®, and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90, and Akypoquat® 131 are examples of these esterquats.

Other compounds of the formula (Tkat1-2) that are particularly preferred according to the invention include the cationic betaine esters of formula (Tkat2.1).

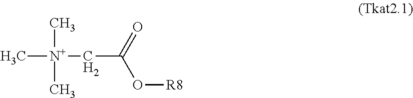

R8 corresponds in its meaning to R7.

Particularly preferred are the esterquats with the trade names Armocare® VGH-70, as well as Dehyquart® F-75, Dehyquart® L80, Stepantex® VS 90, and Akypoquat® 131.

In preferred compositions (c) of the invention, cationic surfactants of the formula (b1) are used within rather narrow amount ranges, so that preferred compositions (c) of the invention are characterized in that they include 0.1 to 15% by weight, preferably 0.5 to 10% by weight, more preferably 1 to 10% by weight, even more preferably 1.5 to 10% by weight, and in particular 2 to 5% by weight of at least one compound of the general formula (I), based on the weight of the composition (c).

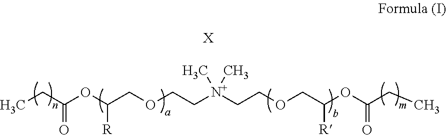

where
n and m independently of one another stand for integers between 5 and 40, with the proviso that n+m≧38; n=m is especially preferred; n=m=20 is most preferable.
a and b independently of one another stand for integers between 1 and 10; particularly independently of one another stand for 1, 2, 3, 4, or 5, preferably the equation a+2≧b≧a−2 applies here and most preferably a=b=3.
R and R′ independently of one another are selected from —H and —CH₃; preferably R=R′, so that preferably either PEG or PPG diesterquats are used; very particularly preferably R=R′=—CH₃.
X⁻ is a physiologically acceptable anion, a halide such as chloride, bromide, or iodide, toluenesulfonate, methosulfate, etc., and particularly preferably methosulfate.

In particular when one the compounds of the formula (I) as previously described is used, it has been found that the care effects of compositions (c) of the invention can be increased further and in particular the stability of the agents can be improved further, if the agents include certain acylated diamines in addition to the compound(s) of the formula (I).

Preferred compositions (c) of the invention are therefore characterized in that they include in addition 0.1 to 10% by weight of at least one compound of the formula (II)

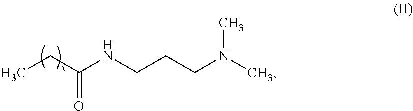

where x stands for 18, 19, 20, 21, 22, 23, or 24.

Compounds of the formula (II) with n=20 are particularly preferred. Most preferred compositions (c) of the invention are characterized in that they always include a compound of the formula (I) together with a compound of the general formula (II).

Another group constitutes quaternary imidazoline compounds. The formula (Tkat2) illustrated below shows the structure of these compounds.

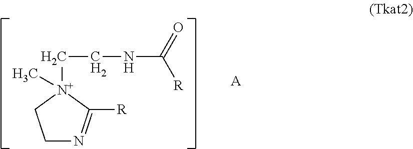

The R groups independently of one another each stand for a saturated or unsaturated, linear or branched hydrocarbon group with a chain length of 8 to 30 carbon atoms. The preferred compounds of the formula (Tkat2) include in each case the same hydrocarbon group for R. The chain length of the groups R is preferably 12 to 21 carbon atoms. ‘A’ stands for an anion as described previously. Particularly inventive examples are obtainable, for example, under the INCI names: Quaternium-27, Quaternium-72, Quaternium-83, and Quaternium-91. Quaternium-91 is most preferable according to the invention.

In a particularly preferred embodiment of the invention, compositions (c) of the invention include furthermore at least one amine and/or cationized amine, in particular an amidoamine and/or a cationized amidoamine of the following structural formulas:

R1-NH—(CH₂)ₙ—N⁺R²R³R⁴A (Tkat3)

where R1 denotes an acyl or alkyl group having 6 to 30 C atoms, which can be branched or unbranched, saturated or unsaturated, and whereby the acyl group and/or alkyl residue can include at least one OH group, and
R2, R3 and R4 in each case independently of one another denote
1) hydrogen or
2) an alkyl group having 1 to 4 C atoms, which may be identical or different, saturated or unsaturated, and
3) a branched or unbranched hydroxyalkyl group having 1 to 4 carbon atoms with at least one and at most three hydroxy groups, for example, —CH₂OH, —CH₂CH₂OH, —CHOHCHOH, —CH₂CHOHCH₃, —CH(CH₂OH)₂, COH(CH₂OH)₂, —CH₂CHOHCH₂OH, —CH₂CH₂CH₂OH, and hydroxybutyl groups, and
   ‘A’ an anion as previously described, and
   n an integer between 1 and 10.

A composition is preferred in which the amine and/or the quaternized amine according to general formula (Tkat3) is an amidoamine and/or a quaternized amidoamine in which R1 denotes a branched or unbranched, saturated or unsaturated acyl group having 6 to 30 C atoms, which can include at least one OH group. A fatty acid group from oils and waxes, in particular from natural oils and waxes, is preferred here. Suitable examples include lanolin, beeswax or candelilla wax.

Also preferred are amidoamines and/or quaternized amidoamines in which R2, R3 and/or R4 in the formula (Tkat3) denote a group according to the general formula CH₂CH₂OR5, where R5 can have the meaning of alkyl groups having 1 to 4 carbon atoms, hydroxyethyl, or hydrogen. The preferred value of n in the general formula (Tkat8) is an integer between 2 and 5.

The alkyl amidoamines can be present as is and can be converted into a quaternary compound in the composition by protonation in a suitably acid solution. Cationic alkyl amidoamines are preferred according to the invention.

Examples of such commercial products of the invention are Witcamine 100, Incromine® BB, Mackin® 401 and other Mackine® types, Adogen® S18V, and as permanently cationic aminoamines: Rewoquat® RTM 50, Empigen® CSC, Swanol® Lanoquat DES-50, Rewoquat® UTM 50, Schercoquat® BAS, Lexquat® AMG-BEO, or Incroquat® Behenyl HE.

A further fatty acid amide of the invention corresponds to the general formula (I).

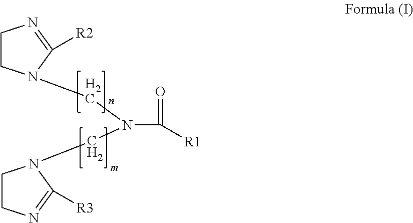

where R1, R2, and R3 independently of one another stand for a linear branched or unbranched C6 to C30, preferably C8 to C24, more preferably C12 to C22, and most preferably C12 to C18 alkyl or alkenyl group. R1 to R3 preferably stand for capryl, caprylyl, octyl, nonyl, decanyl, lauryl, myristyl, cetyl, stearyl, isostearyl, oleyl, behenyl, or arachidyl. Furthermore, particularly preferably R2 is the same as R3 and most preferably R1 is the same as R2 and the same as R3. The letters n and m independently of one another stand for integers from 1 to 10, preferably 2 to 6, and most preferably for 2, 3, and/or 4, whereby n=m is most preferable. Most preferably, R1 is the same as R2 is the same as R3 and is selected from capryl, caprylyl, octyl, nonyl, decanyl, lauryl, myristyl, cetyl, stearyl, isostearyl, oleyl, behenyl, or arachidyl and n=m=2. Most preferably, R1=R2=R3 and is selected from lauryl, myristyl, cetyl, stearyl, isostearyl, oleyl, behenyl or arachidyl, among which cetyl, stearyl, isostearyl, oleyl, or behenyl are particularly preferred and n=m=2. The most preferred compound of the formula (I) is the one with the INCI name Bis-ethyl(isostearylimidazoline) Isostearamide. The last compound is commercially available under the trade name Keradyn® HH from the company Croda.

The aforesaid cationic surfactants can be used individually or in any combinations with one another, whereby they are included in amounts between 0.01 to 10% by weight, preferably in amounts from 0.01 to 7.5% by weight, and very particularly preferably in amounts of 0.1 to 5.0% by weight. The very best results in this case are obtained with amounts of 0.1 to 3.0% by weight, in each case based on the total composition of the particular compositions (c).

Further quaternary ammonium compound are cationic and amphoteric polymers.

The cationic and/or amphoteric polymers can be homopolymers or copolymers or polymers based on natural polymers, whereby the quaternary nitrogen groups can be included either in the polymer chain or preferably as a substituent on one or more of the monomers. The ammonium group-containing monomers can be copolymerized with non-cationic monomers. Suitable cationic monomers are unsaturated, radically polymerizable compounds bearing at least one cationic group, in particular ammonium-substituted vinyl monomers such as, for example, trialkyl methacryloxy alkylammonium, trialkyl acryloxy alkylammonium, dialkyl diallyl ammonium, and quaternary vinyl ammonium monomers with cyclic groups containing cationic nitrogens, such as pyridinium, imidazolium, or quaternary pyrrolidones, e.g., alkyl vinylimidazolium, alkyl vinylpyridinium, or alkyl vinylpyrrolidone salts. The alkyl groups of these monomers are preferably low alkyl groups such as, for example, C1 to C7 alkyl groups, particularly preferably C1 to C3 alkyl groups.

The ammonium group-containing monomers can be copolymerized with non-cationic monomers. Suitable comonomers are, for example, acrylamide, methacrylamide, alkyl and dialkyl acrylamide, alkyl and dialkyl methacrylamide, alkyl acrylate, alkyl methacrylate, vinyl caprolactone, vinyl caprolactam, vinylpyrrolidone, vinyl esters, e.g., vinyl acetate, vinyl alcohol, propylene glycol, or ethylene glycol, the alkyl groups of these monomers preferably being C1 to C7 alkyl groups, particularly preferably C1 to C3 alkyl groups.

From the large number of these polymers, the following proved to be particularly effective components of the active agent complex of the invention:

homopolymers of the general formula —{CH₂—[CR¹COO—(CH₂)ₘN⁺R²R³R⁴]}ₙX⁻,
where R′=—H or —CH3, R², R³, and R⁴ independently of one another are selected from C1-4 alkyl, alkenyl, or hydroxyalkyl groups, m=1, 2, 3, or 4, n is a natural number, and
X⁻ is a physiologically acceptable organic or inorganic anion. In the context of these polymers, those for which at least one of the following conditions applies are preferred according to the invention: R¹ denotes a methyl group, R², R³, and R⁴ stand for methyl groups, and m has the value 2.

Suitable physiologically acceptable counterions X⁻ are, for example, halide ions, sulfate ions, phosphate ions, methosulfate ions, as well as organic ions such as lactate, citrate, tartrate, and acetate ions. Methosulfates and halide ions, in particular chloride, are preferred.

Suitable cationic polymers are, for example, copolymers according to the formula (Copo), which are preferably included in compositions (c) of the invention in an amount, based on their weight, of 0.001 to 5% by weight, preferably 0.0025 to 2.5% by weight, particularly preferably 0.005 to 1% by weight, more preferably 0.0075 to 0.75% by weight, and in particular 0.01 to 0.5% by weight.

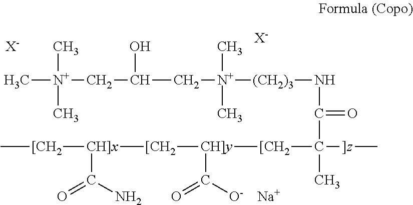

where:
x+y+z=Q
Q stands for values from 3 to 55,000, preferably from 10 to 25,000, particularly preferably from 50 to 15,000, more preferably from 100 to 10,000, even more preferably from 500 to 8000, and in particular from 1000 to 5000,
x stands for (0 to 0.5) Q, preferably for (0 to 0.3) Q, and in particular for the values 0, 1, 2, 3, 4, 5, the value 0 being preferred,
y stands for (0.1 to 0.95) Q, preferably for (0.5 to 0.7) Q, and in particular for values from 1 to 24,000, preferably from 5 to 15,000, particularly preferably from 10 to 10,000, and in particular from 100 to 4800,
z stands for (0.001 to 0.5) Q, preferably for (0.1 to 0.5) Q, and in particular for values from 1 to 12,500, preferably from 2 to 8000, particularly preferably from 3 to 4000, and in particular from 5 to 2000.

Regardless of which of the preferred copolymers of the formula (Copo) are used, hair treatment agents of the invention are preferred that are characterized in that the ratio of (y:z) is 4:1 to 1:2, preferably 4:1 to 1:1.

Regardless of which copolymers are used in compositions (c) of the invention, compositions (c) of the invention are preferred in which the copolymer has a molar mass of 10,000 to 20 million gmol⁻¹, preferably of 100,000 to 10 million gmol⁻¹, more preferably of 500,000 to 5 million gmol⁻¹, and in particular of 1.1 million to 2.2 million gmol⁻¹.

A highly preferred copolymer, which has the structure as described above, is commercially available under the name Polyquaternium-74.

A particularly suitable homopolymer is the optionally crosslinked poly(methacryloyloxyethyl trimethylammonium chloride) with the INCI name Polyquaternium-37. Such products are commercially available, for example, under the names Rheocare® CTH (Cosmetic Rheologies) and Synthalen® CR (3V Sigma).

The homopolymer is preferably used in the form of a nonaqueous polymer dispersion. Such polymer dispersions are commercially available under the names Salcare® SC 95 and Salcare® SC 96.

Suitable cationic polymers that are derived from natural polymers are cationic derivatives of polysaccharides, for example, cationic derivatives of cellulose, starch, or guar. Also suitable are chitosan and chitosan derivatives. Cationic polysaccharides have the general formula G-O—B—N⁺RₐR_{b}R_{c}A⁻:

G is an anhydroglucose group, for example, starch or cellulose anhydroglucose;
B is a divalent connecting group, for example, alkylene, oxyalkylene, polyoxyalkylene, or hydroxyalkylene;
Rₐ, R_{b}, and R_{c} are independently of one another alkyl, aryl, alkylaryl, arylalkyl, alkoxyalkyl, or alkoxyaryl, each having up to 18 C atoms, the total number of C atoms in
Rₐ, R_{b}, and R_{c} preferably being a maximum of 20;
A⁻ is a conventional counteranion and is preferably chloride.

Cationic, therefore, quaternized, celluloses are available on the market with varying degrees of substitution, cationic charge density, nitrogen content, and molecular weights. For example, Polyquaternium-67 is commercially available under the names Polymer® SL or Polymer® SK (Amerchol). A further most highly preferred cellulose is available from the company Croda under the trade name Mirustyle® CP. This is a trimonium and cocodimonium hydroxyethylcellulose as a derivatized cellulose with the INCI name Polyquaternium-72. Polyquaternium-72 can be used both in solid form and already pre-dissolved in aqueous solution.

Further cationic celluloses are Polymer JR® 400 (Amerchol, INCI name Polyquaternium-10) and Polymer Quatrisoft® LM-200 (Amerchol, INCI name Polyquaternium-24). Further commercial products are the compounds Celquat® H 100 and Celquat® L 200. Particularly preferred cationic celluloses are Polyquaternium-24, Polyquaternium-67, and Polyquaternium-72.

Suitable cationic guar derivatives are sold under the trade name Jaguar and have the INCI name Guar Hydroxypropyltrimonium Chloride. Particularly suitable cationic guar derivatives are also sold furthermore by the company Hercules under the name N-Hance®. Further cationic guar derivatives are sold by the company Cognis under the name Cosmedia®. A preferred cationic guar derivative is the commercial product AquaCat® from the company Hercules. This raw material is an already pre-dissolved cationic guar derivative. The cationic guar derivatives are preferred according to the invention.

A suitable chitosan is sold, for example, by the company Kyowa Oil & Fat, Japan, under the trade name Flonac®. A preferred chitosan salt is chitosonium pyrrolidone carboxylate, which is sold, for example, under the name Kytamer® PC by the company Amerchol, USA. Further chitosan derivatives are freely available commercially under the trade names Hydagen® CMF, Hydagen® HCMF, and Chitolam® NB/101.

A further group of polymers to be used excellently according to the invention are glucose-based polymers. The following figure shows such a cationic alkyl oligoglucoside.

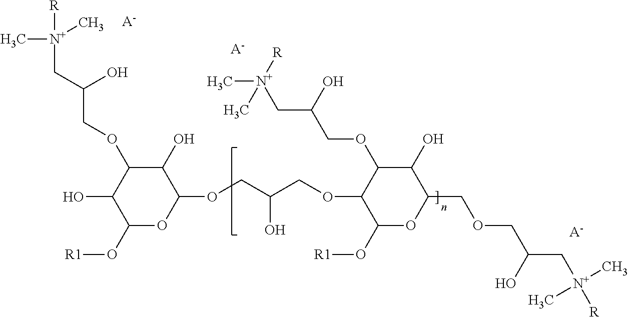

In the above formula the R groups independently of one another stand for a linear or branched C6 to C30 alkyl group, a linear or branched C6 to C30 alkenyl group; the R group preferably stands for an R group selected from: lauryl, myristyl, cetyl, stearyl, oleyl, behenyl, or arachidyl.

The R1 groups independently of one another stand for a linear or branched C6 to C30 alkyl group, a linear or branched C6 to C30 alkenyl group; the R group preferably stands for a group selected from: butyl, capryl, caprylyl, octyl, nonyl, decanyl, lauryl, myristyl, cetyl, stearyl, oleyl, behenyl, or arachidyl. Particularly preferably the R1 groups are the same. Still more preferably, the R1 groups are selected from technical mixtures of the fatty alcohol cuts from C6/C8 fatty alcohols, C8/C10 fatty alcohols, C10/C12 fatty alcohols, C12/C14 fatty alcohols, and C12/C18 fatty alcohols, and the technical fatty alcohol cuts that are of plant origin are most preferred. The counterion for cationic charging is a physiologically acceptable anion, for example, halide, methosulfate, phosphate, citrate, tartrate, etc. The counterion is preferably a halide, such as fluoride, chloride, bromide, or methosulfate. The anion is most preferably chloride.

Particularly preferred examples for the cationic alkyl oligoglucosides are the compounds with the INCI names: Polyquaternium-77, Polyquaternium-78, Polyquaternium-79, Polyquaternium-80, Polyquaternium-81, and Polyquaternium-82. Most preferred are the cationic alkyl oligoglucosides with the names Polyquaternium-77, Polyquaternium-81, and Polyquaternium-82.

Such compounds can be obtained, for example, from the company Colonial Chemical Inc. under the name Poly Suga® Quat.

The cationic alkyl oligoglucosides are used in a total amount of 0.01 to 10.0% by weight, preferably of 0.05 to 5.0% by weight, more preferably of 0.1 to 3.0% by weight, and most preferably in amounts of 0.2 to 2.0% by weight, based in each case on the total weight of composition (c). The invention, of course, also comprises the fact that mixtures of cationic alkyl oligoglucosides can be used. In this case it is preferable for a long-chain and a short-chain cationic alkyl oligoglucoside to be used at the same time in each case.

A further preferred cationic polymer can be obtained on the basis of ethanolamine. The polymer is commercially available under the name Polyquaternium-71.

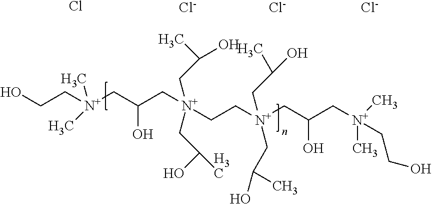

This polymer can be obtained, for example, from the company Colonial Chemical Inc. under the name Cola® Moist 300 P.

Polyquaternium-71 is used in a total amount of 0.01 to 10.0% by weight, preferably of 0.05 to 5.0% by weight, more preferably of 0.1 to 3.0% by weight, and most preferably of 0.2 to 2.0% by weight, based in each case to the total weight of composition (c).

Furthermore, a cationic alkyl oligoglucoside as shown in the following figure can be used to particular advantage.

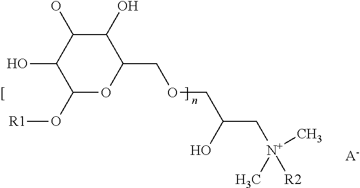

In the above formula, the R2 group stands for a linear or branched C6 to C30 alkyl group, a linear or branched C6 to C30 alkenyl group; the R group preferably stands for an R group selected from: lauryl, myristyl, cetyl, stearyl, oleyl, behenyl, or arachidyl.

The R1 group stands for a linear or branched C6 to C30 alkyl group, a linear or branched C6 to C30 alkenyl group; the R1 group preferably stands for a group selected from: butyl, capryl, caprylyl, octyl, nonyl, decanyl, lauryl, myristyl, cetyl, stearyl, oleyl, behenyl, or arachidyl. Still more preferably, the R1 group is selected from technical mixtures of the fatty alcohol cuts from C6/C8 fatty alcohols, C8/C10 fatty alcohols, C10/C12 fatty alcohols, C12/C14 fatty alcohols, and C12/C18 fatty alcohols, and most preferred here are the technical fatty alcohol cuts that are of plant origin. The subscript n stands for a number between 1 and 20, preferably between 1 and 10, more preferably between 1 and 5, and most preferably between 1 and 3. The counterion for cationic charging, A⁻, is a physiologically acceptable anion, for example, halide, methosulfate, phosphate, citrate, tartrate, etc. The counterion is preferably a halide, such as fluoride, chloride, bromide, or methosulfate. The anion is most preferably chloride.

Particularly preferred examples for the cationic alkyl oligoglucosides are the compounds with the INCI names Laurdimoniumhydroxypropyl Decylglucosides Chloride, Laurdimoniumhydroxypropyl Laurylglucosides Chloride, Stearyldimoniumhydroxypropyl Decylglucosides Chloride, Stearyldimoniumhydroxypropyl Laurylglucosides Chloride, Stearyldimoniumhydroxypropyl Laurylglucosides Chloride, or Cocoglucosides Hydroxypropyltrimonium Chloride.

Such compounds can be obtained, for example, from the company Colonial Chemical Inc. under the name Suga® Quat.

The cationic alkyl oligoglucosides are used in a total amount of 0.01 to 10.0% by weight, preferably of 0.05 to 5.0% by weight, more preferably of 0.1 to 3.0% by weight, and most preferably in amounts of 0.2 to 2.0% by weight, based in each case on the total weight of composition (c). The invention, of course, also comprises the fact that mixtures of cationic alkyl oligoglucosides can be used. In this case it is preferable for a long-chain and a short-chain cationic alkyl oligoglucoside to be used at the same time in each case.

The polymers described thus far represent only part of the polymers that can be used according to the invention. In order not to have to describe all of the cationic and/or amphoteric polymers suitable according to the invention, in addition to their composition, the INCI declarations of the polymers preferred according to the invention are listed as a summary. The polymers preferred according to the invention have the INCI name:

Polyquaternium-4, Polyquaternium-6, Polyquaternium-15, Polyquaternium-16, Polyquaternium-22, Polyquaternium-24, Polyquaternium-28, Polyquaternium-32, Polyquaternium-33, Polyquaternium-34, Polyquaternium-35, Polyquaternium-39, Polyquaternium-41, Polyquaternium-42, Polyquaternium-44, Polyquaternium-47, Polyquaternium-55, Polyquaternium-68, Polyquaternium-76, Polyquaternium-86, Polyquaternium-89, and Polyquaternium-95, and mixtures thereof.
Other preferred cationic polymers are, for example,
polymeric dimethyldiallylammonium salts and the copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride/acrylamide copolymer) are examples of such cationic polymers with the INCI name Polyquaternium-7,
vinyl pyrrolidone/vinyl imidazolium methochloride copolymers, as are offered under the names Luviquat® FC 370, FC 550, and the INCI name Polyquaternium-16, as well as FC 905 and HM 552,
quaternized vinyl pyrrolidone/dimethylaminoethyl methacrylate, for example, vinyl pyrrolidone/dimethylaminoethyl methacrylate methosulfate copolymer, which is marketed under the trade names Gafquat® 755 N and Gafquat® 734 by the company Gaf Co., USA, and the INCI name Polyquaternium-11,
quaternized polyvinyl alcohol,
as well as the polymers having quaternary nitrogen atoms in the main polymer chain, known under the names Polyquaternium-2, Polyquaternium-17, Polyquaternium-18, and Polyquaternium-27,
vinyl pyrrolidone/vinyl caprolactam/acrylate terpolymers, as are commercially available with acrylic acid esters and acrylamides as the third monomeric unit, for example, under the name Aquaflex® SF 40.

The aforementioned cationic polymers can be used individually or in any combinations with one another, whereby amounts between 0.01 to 10% by weight, preferably amounts of 0.01 to 7.5% by weight, and very particularly preferably in amounts of 0.1 to 5.0% by weight are included. The very best results in this case are obtained with amounts of 0.1 to 3.0% by weight, based in each case on the total composition the particular compositions (c).

Amphoteric polymers of the invention are polymers in which a cationic group is derived from at least one of the following monomers:
(i) monomers with quaternary ammonium groups of the general formula (Mono1),

R¹—CH═CR²—CO—Z—(CₙH₂ₙ)—N⁽⁺⁾R²R³R⁴A⁽⁻⁾ (Mono1)
in which R¹ and R² independently of one another stand for hydrogen or a methyl group and R³, R⁴, and R⁵ independently of one another for alkyl groups having 1 to 4 carbon atoms, Z is an NH group or an oxygen atom, n is an integer from 2 to 5, and A⁽⁻⁾ is the anion of an organic or inorganic acid,
(ii) monomers with quaternary ammonium groups of the general formula (Mono2),

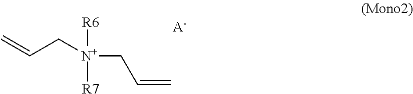

where R⁶ and R⁷ independently of one another stand for a (C₁ to C₄) alkyl group, in particular for a methyl group, and
A⁻ is the anion of an organic or inorganic acid,
(iii) monomeric carboxylic acids of the general formula (Mono3),

R⁸—CH═CR⁹—COOH (Mono3)
where R⁸ and R⁹ independently of one another are hydrogen or methyl groups.

Particularly preferred are those polymers in which monomers of type (i) are used in which R³, R⁴, and R⁵ are methyl groups, Z is an NH group, and A⁽⁻⁾ is a halide, methoxysulfate, or ethoxysulfate ion; (acrylamidopropyl)trimethylammonium chloride is a particularly preferred monomer (i). Acrylic acid is used preferably as monomer (ii) for the named polymers.

Particularly preferred amphoteric polymers are copolymers of at least one monomer (Mono1) or (Mono2) with the monomer (Mono3), in particular copolymers of monomers (Mono2) and (Mono3). Amphoteric polymers that are used very particularly preferably according to the invention are copolymers of diallyldimethylammonium chloride and acrylic acid. These copolymers are sold under the INCI name Polyquaternium-22, inter alia, under the trade name Merquat® 280 (Nalco).

In addition to a monomer (Mono1) or (Mono2) and a monomer (Mono3), the amphoteric polymers of the invention can, moreover, additionally include a monomer (Mono4)
(iv) monomeric carboxylic acid amides of the general formula (Mono4),

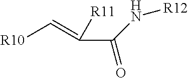

where R¹⁰ and R¹¹ independently of one another are hydrogen or methyl groups and R¹² stands for a hydrogen atom or a (C₁ to C₈) alkyl group.

Amphoteric polymers based on a comonomer (Mono4) that are used very particularly preferably according to the invention are terpolymers of diallyldimethylammonium chloride, acrylamide, and acrylic acid. These copolymers are sold under the INCI name Polyquaternium-39, inter alia, under the trade name Merquat® Plus 3330 (Nalco).

The amphoteric polymers can generally be used according to the invention both directly and in the form of the salt, obtained by neutralization of the polymers, for example, with an alkali hydroxide.

The aforementioned cationic polymers can be used individually or in any combinations with one another, whereby they are contained in amounts between 0.01 and 10% by weight, preferably in amounts of 0.01 to 7.5% by weight, and very particularly preferably in amounts of 0.1 to 5.0% by weight. The very best results in this case are obtained with amounts of 0.1 to 3.0% by weight, based in each case on the total composition the particular compositions (c).

The mixing ratio of compositions (a), (b), and (c) is selected as follows: Compositions (a) and (b) are mixed with one another in the ratio of 1:1. Added to the mixture of compositions (a) and (b) are 0.01 to 25% by weight of composition (c), preferably 0.5 to 23.0% by weight, more preferably 0.5 to 15.0% by weight, and most preferably 4 to 12.0% by weight, relative to the sum of the total weights of compositions (a) and (b).

In a further embodiment of the present invention, therefore agents are preferred that are characterized in that they are prepared immediately before use by mixing at least three preparations, whereby the at least three preparations are provided in at least three separately produced containers, and whereby one container includes a composition (a), which includes at least one dye precursor in a cosmetic carrier, a further container includes an oxidizing agent preparation (b), including at least one oxidizing agent, and at least one third container includes a composition (c) with a customary conditioning composition.

Optionally, direct dyes and/or oxidation dye precursors of the developer and/or coupler type, contained in addition, are produced in this case advantageously together with composition (a) of the invention.

Compositions (a) to (c) of the invention include a cosmetic carrier. Aqueous, alcoholic, or aqueous-alcoholic carriers are used preferentially as cosmetic carriers. For the purpose of dyeing hair, such carriers can be, for example, creams, emulsions, gels, or surfactant-containing foaming solutions as well, such as, for example, shampoos, foam aerosols, foam formulations, or other preparations suitable for use on hair. It is also conceivable, however, to integrate the agents of the invention into a powdery or also tablet-like preparation.

Aqueous-alcoholic solutions in the context of the present invention are understood to be aqueous solutions including 3 to 70% by weight of a C₁-C₄ alcohol, particularly ethanol or isopropanol. The agents of the invention can include in addition further organic solvents such as, for example, methoxybutanol, benzyl alcohol, ethyl diglycol, or 1,2-propylene glycol. In this case, all water-soluble organic solvents are preferred.

The dye preparation (a), the oxidizing agent preparation (b), and the conditioning preparation (c) include further auxiliary substances and additives. Thus, it has proven preferable according to the invention, if the preparations include at least one thickener. There are no basic restrictions with regard to these thickeners. Both organic and purely inorganic thickeners may be used.

According to a first preferred embodiment, the thickener is an anionic, synthetic polymer. Preferred anionic groups are the carboxylate and the sulfonate group.

Examples of anionic monomers of which the polymeric anionic thickeners can consist are acrylic acid, methacrylic acid, crotonic acid, itaconic acid, maleic anhydride, and 2-acrylamido-2-methylpropanesulfonic acid. In this regard, the acid groups can be present entirely or partially as the sodium, potassium, ammonium, or the mono- or triethanolammonium salt. Preferred monomers are maleic anhydride and in particular 2-acrylamido-2-methylpropanesulfonic acid and acrylic acid.

In the context of work leading to this invention, it was found that advantageous results were obtained particularly when the agent of the invention includes in addition at least one anionic acrylic acid polymer and/or an anionic acrylic acid copolymer.

The anionic acrylic acid polymer and/or anionic acrylic acid copolymer in this case can be present in the agent of the invention in a weight proportion of 0.001 to 20%, preferably up to 0.01 to 10%, and particularly preferably of 0.5 to 5%.

A further preferred embodiment of the first subject of the invention, therefore, is an agent of the invention, which is characterized in that it includes in addition, based on its weight, 0.001 to 20% of at least one anionic acrylic acid polymer and/or an anionic acrylic acid copolymer.

It may be preferred in this connection if the anionic acrylic acid copolymer additionally present in the agent of the invention is a copolymer that is prepared by copolymerization of the monomers acrylic acid (prop-2-enoic acid), methacrylic acid (2-methylprop-2-enoic acid), acrylic acid methyl ester (methyl prop-2-enoate), methacrylic acid methyl ester (methyl 2-methylprop-2-enoate), acrylic acid ethyl ester (ethyl prop-2-enoate), methacrylic acid ethyl ester (ethyl 2-methylprop-2-enoate), acrylic acid-n-butyl ester (butyl prop-2-enoate), methacrylic acid-n-butyl ester (butyl 2-methylprop-2-enoate), ethene, and/or styrene (ethenylbenzene).

According to a preferred embodiment, the agent of the invention additionally includes an anionic acrylic acid copolymer that is prepared by copolymerization of the monomers methacrylic acid (2-methylprop-2-enoic acid), methacrylic acid methyl ester (methyl 2-methylprop-2-enoate), acrylic acid ethyl ester (ethyl prop-2-enoate), acrylic acid-n-butyl ester (butyl prop-2-enoate), ethene, and/or styrene (ethenylbenzene).

According to a very particularly preferred embodiment, the agent of the invention additionally includes an anionic acrylic acid copolymer that is prepared by copolymerization of the monomers methacrylic acid (2-methylprop-2-enoic acid), methacrylic acid methyl ester (methyl 2-methylprop-2-enoate), acrylic acid ethyl ester (ethyl prop-2-enoate), acrylic acid-n-butyl ester (butyl prop-2-enoate), ethene, and styrene (ethenylbenzene).

A particularly preferred anionic acrylic acid copolymer is the one referred to by the INCI name Polyacrylate-15.

A further particularly preferred embodiment of the first subject of the invention is therefore an agent of the invention that is characterized in that it includes Polyacrylate-15 as the anionic acrylic acid copolymer.

In a further embodiment, the agent of the invention may include anionic homopolymers as anionic polymers. Preferred anionic homopolymers are non-crosslinked and crosslinked polyacrylic acids. In this regard, allyl ethers of pentaerythritol, of sucrose, and of propylene can be preferred crosslinking agents. Such compounds are, for example, available commercially under the trademark Carbopol®. Likewise preferred is the homopolymer of 2-acrylamido-2-methylpropanesulfonic acid, which is available commercially, for example, under the name Rheothik® 11-80.

Within a further embodiment it may likewise be preferred to employ copolymers of at least one anionic monomer and at least one nonionogenic monomer. In regard to the anionic monomers, reference is made to the substances cited above. Preferred nonionogenic monomers are acrylamide, methacrylamide, acrylic acid esters, methacrylic acid esters, itaconic acid mono- and diesters, vinylpyrrolidone, vinyl ethers, and vinyl esters.

Other preferred anionic copolymers are, for example, copolymers of acrylic acid, methacrylic acid, or the C₁-C₆ alkyl esters thereof, as they are sold under the INCI declaration Acrylates Copolymer. A preferred commercial product is, for example, Aculyn® 33 from the company Rohm & Haas. Preferred further, however, are also copolymers of acrylic acid, methacrylic acid, or the C₁-C₆ alkyl esters thereof and the esters of an ethylenically unsaturated acid and an alkoxylated fatty alcohol. Suitable ethylenically unsaturated acids are in particular acrylic acid, methacrylic acid, and itaconic acid; suitable alkoxylated fatty alcohols are in particular Steareth-20 or Ceteth-20. Copolymers of this type are sold by Rohm & Haas under the trade name Aculyn® 22 and by the company National Starch under the trade names Structure® 2001 and Structure® 3001.

Preferred anionic copolymers are furthermore acrylic acid/acrylamide copolymers and, in particular, polyacrylamide copolymers having sulfonic acid group-containing monomers. A particularly preferred anionic copolymer consists of 70 to 55 mol % of acrylamide and 30 to 45 mol % of 2-acrylamido-2-methylpropanesulfonic acid, the sulfonic acid group being present entirely or in part as a sodium, potassium, ammonium, or mono- or triethanolammonium salt. This copolymer may also be present crosslinked, whereby polyolefinically unsaturated compounds such as tetraallyloxyethane, allyl sucrose, allyl pentaerythritol, and methylene bisacrylamide are preferably employed as crosslinking agents. One such polymer is included in the commercial products Sepigel® 305 polymer and Simugel® 600 from the company Seppic. The use of these compounds, which, besides the polymer component, include a hydrocarbon mixture (C₁₃ to C₁₄ isoparaffin or isohexadecane) and a nonionic emulsifier (Laureth-7 or Polysorbate-80), has proven particularly advantageous in the context of the teaching of the invention.

Polymers of maleic anhydride and methyl vinyl ether, in particular those having crosslinks, are also preferred thickeners. A maleic acid/methyl vinyl ether copolymer crosslinked with 1,9-decadiene is obtainable commercially under the name Stabileze® QM.

The agent of the present invention may preferably additionally include at least one further anionic polymer or copolymer of acrylic acid and/or methacrylic acid. Preferred polymers of this kind are:
polymers of, e.g., at least 10% by weight of acrylic acid-low alkyl esters, 25 to 70% by weight of methacrylic acid, and, optionally, up to 40% by weight of a further comonomer,
mixed polymers of 50 to 75% by weight of ethyl acrylate, 25 to 35% by weight of acrylic acid, and 0 to 25% by weight of further comonomers. Suitable dispersions of this kind are commercially obtainable, e.g., under the trade name Latekoll® D (BASF).
copolymers of 50 to 60% by weight of ethyl acrylate, 30 to 40% by weight of methacrylic acid, and 5 to 15% by weight of acrylic acid, crosslinked with ethylene glycol dimethacrylate.

According to a further embodiment, the thickener is a cationic synthetic polymer that differs from the cationic polymers of the invention. Preferred cationic polymers are:
homopolymers of the general formula (HP-1),

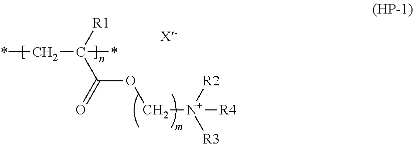

where R1=—H or —CH3 is, R2, R3, and R4 independently of one another are selected from C1-C4 alkyl, alkenyl, or hydroxyalkyl groups, m=1, 2, 3, or 4, n is a natural number, and X⁻ is a physiologically acceptable organic or inorganic anion, are particularly preferred cationic polymeric gelling agents. In the context of these polymers, those are preferred according to the invention for which at least one of the following conditions applies:
R1 stands for a methyl group
R2, R3, and R4 stand for methyl groups
m has the value of 2.

Appropriate physiologically acceptable counterions X′ are, for example, halide ions, sulfate ions, phosphate ions, methosulfate ions, and organic ions such as lactate, citrate, tartrate, and acetate ions. Halide ions, in particular chloride, are preferred.

A particularly suitable homopolymer is the optionally crosslinked poly(methacryloxyethyltrimethylammonium) chloride having the INCI name Polyquaternium-37. The crosslinking may occur, if desired, with the aid of olefinically unsaturated compounds, for example, divinylbenzene, tetraallyloxyethane, methylene bisacrylamide, diallyl ether, polyallyl polyglyceryl ether, or allyl ethers of sugars, or sugar derivatives such as erythritol, pentaerythritol, arabitol, mannitol, sorbitol, sucrose, or glucose. Methylene bisacrylamide is a preferred crosslinking agent.

The homopolymer is preferably used in the form of a nonaqueous polymer dispersion that should have a polymer proportion not less than 30% by weight. Such polymer dispersions are obtainable commercially under the names Salcare® SC 95 (approximately 50% polymer proportion, further components: mineral oil (INCI name: Mineral Oil) and tridecyl polyoxypropylene polyoxyethylene ether (INCI name: PPG-1 Trideceth-6)), and Salcare® SC 96 (approximately 50% polymer proportion, further components: mixture of diesters of propylene glycol with a mixture of caprylic and capric acid (INCI name: Propylene Glycol Dicaprylate/Dicaprate), and tridecyl polyoxypropylene polyoxyethylene ether (INCI name: PPG-1 Trideceth-6)).

In a further preferred embodiment, naturally occurring thickeners are used. Preferred thickeners of this embodiment are, for example, nonionic guar gums. Both modified and unmodified guar gums may be employed according to the invention. Unmodified guar gums are marketed, for example, under the trade name Jaguar® C by the company Rhone-Poulenc. Modified guar gums preferred according to the invention include C₁-C₆ hydroxyalkyl groups. The hydroxymethyl, hydroxyethyl, hydroxypropyl, and hydroxybutyl groups are preferred. Guar gums modified in this fashion are known in the prior art and may be prepared, for example, by reacting the guar gums with alkylene oxides. The degree of hydroxyalkylation, which corresponds to the number of alkylene oxide molecules consumed relative to the number of free hydroxy groups in the guar gums, is preferably between 0.4 and 1.2. Guar gums modified in this fashion are obtainable commercially under the commercial names Jaguar® HP8, Jaguar® HP60, Jaguar® HP120, Jaguar® DC 293, and Jaguar® HP105 from the company Firma Rhone Poulenc.

Further suitable natural thickeners are also already known from the prior art.

Also preferred according to this embodiment are biosaccharide gums of microbial origin, such as scleroglucan gums or xanthan gums, gums from plant exudates such as gum arabic, ghatti gum, karaya gum, tragacanth gum, carrageenan gum, agar-agar, locust bean flour, pectins, alginates, starch fractions and derivatives such as amylose, amylopectin, and dextrins, cellulose derivatives, such as, for example, methyl cellulose, carboxyalkyl celluloses, and hydroxyalkyl celluloses.

Preferred hydroxyalkyl celluloses are in particular the hydroxyethyl celluloses marketed under the names Cellosize® by the company Amerchol and Natrosol® by the company Hercules. Suitable carboxyalkyl celluloses are in particular the carboxymethylcelluloses as they are marketed under the names Blanose® by the company Aqualon, Aquasorb® and Ambergum® by the company Hercules, and Cellgon® by the company Montello.

Preferred furthermore are starch and derivatives thereof. Starch is a storage substance of plants which occurs primarily in tubers and roots, in cereal seeds, and in fruits and can be obtained from a large number of plants in high yield. The polysaccharide, which is insoluble in cold water and forms a colloidal solution in boiling water, can be obtained, for example, from potatoes, cassava, sweet potato, maranta, corn, cereal, rice, legumes such as, for example, peas and beans, bananas, or the marrow of certain types of palm (for example sago palm). According to the invention, it is possible to use natural starches obtained from plants and/or chemically or physically modified starches. Modification can be achieved, for example, by introducing various functional groups onto one or more of the hydroxyl groups of the starch. These are usually esters, ethers, or amides of starch having optionally substituted C₁-C₄₀ groups. A corn starch etherified with a 2-hydroxypropyl group is particularly advantageous, as is sold, for example, by the company National Starch under the trade name Amaze®.

Nonionic, fully synthetic polymers, however, such as, for example polyvinyl alcohol or polyvinylpyrrolidone, are also usable as thickeners of the invention. Preferred nonionic, fully synthetic polymers are marketed, for example, by the company BASF under the trade name Luviskol®. Apart from their outstanding thickening properties, nonionic polymers of this kind also make possible an appreciable improvement in the sensory feel of the resulting preparations.

Phyllosilicates (polymeric crystalline sodium disilicates) have proven to be particularly suitable as inorganic thickeners in the context of the present invention. Clays, in particular, magnesium aluminum silicates, such as bentonite, in particular smectites such as montmorillonite or hectorite, which optionally may also be suitably modified, and synthetic phyllosilicates such as, for example, the magnesium phyllosilicate marketed by the company Süd Chemie under the commercial name Optigel®, are particularly preferred.

The present invention is not limited in principle with regard to the optionally hydrated SiO₂ compounds. Preferred are silicic acids, oligomers and polymers thereof, and salts thereof. Preferred salts are the alkali salts, in particular, the potassium and sodium salts. The sodium salts are very particularly preferred.

The optionally hydrated SiO₂ compounds can be present in various forms. SiO₂ compounds used according to the invention are preferably in the form of silica gels, or particularly preferably as water glass. These SiO₂ compounds can be present in part in aqueous solution.

Very particularly preferred according to the invention are water glasses that are formed from a silicate of the formula (SiO₂)ₙ(Na₂O)ₘ(K₂O)ₚ, where n stands for a positive rational number and m and p, independently of one another, stand for a positive rational number or for 0, with the proviso that at least one of the parameters m or p is different from 0, and that the ratio between n and the sum of m and p is between 1:4 and 4:1.

In addition to the components described by the molecular formula, water glasses can also include further additives such as, for example, phosphates or magnesium salts, in small quantities.

Water glasses particularly preferred according to the invention are marketed, inter alia, by the Henkel company under the names Ferrosil® 119, Natronwasserglas 40/42, Portil® A, Portil® AW, and Portil® W, and by the Akzo company under the name Britesil® C20.

Preferably, furthermore, an emulsifier or surfactant is added to the agent of the invention, whereby surface-active substances are referred to as surfactants or as emulsifiers depending on the field of application and are selected from anionic, cationic, zwitterionic, amphoteric, and nonionic surfactants and emulsifiers. These substances are described in detail below.

All anionic surface-active substances, suitable for use on the human body, are suitable as anionic surfactants in the preparations of the invention. These are characterized by an anionic group imparting water solubility such as, for example, a carboxylate, sulfate, sulfonate, or phosphate group, and a lipophilic alkyl group having, for instance, 8 to 30 C atoms. In addition, the molecule may include glycol ether or polyglycol ether groups, ester, ether, and amide groups, and hydroxyl groups. Examples of suitable anionic surfactants are, each in the form of the sodium, potassium, and ammonium and mono-, di-, and trialkanolammonium salts having 2 to 4 C atoms in the alkanol group,
linear and branched fatty acids having 8 to 30 C atoms (soaps),
ether carboxylic acids of the formula RO(CH₂CH₂O)ₓCH₂COOH, in which R is a linear alkyl group having 8 to 30 C atoms and x=0 or is 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group, and sulfosuccinic acid monoalkylpolyoxyethyl esters having 8 to 24 C atoms in the alkyl group and 1 to 6 oxyethyl groups,
linear alkane sulfonates having 8 to 24 C atoms,
linear α-olefin sulfonates having 8 to 24 C atoms,
sulfonates of unsaturated fatty acids having 8 to 24 C atoms and 1 to 6 double bonds,
α-sulfo fatty acid methyl esters of fatty acids having 8 to 30 C atoms,
alkyl sulfates and alkyl ether sulfates of the formula RO(CH₂CH₂O)ₓOSO₃H, in which R is a preferably linear alkyl group having 8 to 30 C atoms and x=0 or is 1 to 12,
mixtures of surface-active hydroxysulfonates,
sulfated hydroxyalkylpolyethylene and/or hydroxyalkylenepropylene glycol ethers,
esters of tartaric acid and citric acid with alcohols, representing adducts of, for instance, 2 to 15 molecules of ethylene oxide and/or propylene oxide to fatty alcohols having 8 to 22 C atoms,
alkyl and/or alkenyl ether phosphates of the formula

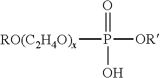

where R preferably stands for an aliphatic, optionally unsaturated hydrocarbon group having 8 to 30 carbon atoms, R′ for hydrogen, a group (CH₂CH₂O)_{y}R, and x and y independently of one another for a number of 1 to 10,
sulfated fatty acid alkylene glycol esters of the formula RC(O)O(alkO)ₙSO₃H, where R stands for a linear or branched, aliphatic, saturated and/or unsaturated alkyl group having 6 to 22 C atoms, alk for CH₂CH₂, CHCH₃CH₂, and/or CH₂CHCH₃, and n for a number of 0.5 to 5,
monoglyceride sulfates and monoglyceride ether sulfates.

Preferred anionic surfactants are alkyl sulfates, alkyl ether sulfates, and ether carboxylic acids having 10 to 18 C atoms in the alkyl group and up to 12 glycol ether groups in the molecule.

Such surface-active compounds that have at least one quaternary ammonium group and at least one carboxylate, sulfonate, or sulfate group in the molecule are called zwitterionic surfactants. Especially suitable zwitterionic surfactants are the so-called betaines, such as the N-alkyl-N,N-dimethylammonium glycinates, for example, coco alkyl dimethylammonium glycinate and N-acyl-aminopropyl-N,N-dimethylammonium glycinates, for example, coco acylaminopropyldimethylammonium glycinate, and 2-alkyl-3-carboxymethyl-3-hydroxyethyl imidazolines each having 8 to 18 C atoms in the alkyl or acyl group, and coco acylaminoethyl hydroxyethyl carboxymethyl glycinate. A preferred zwitterionic surfactant is the fatty acid amide derivative known under the INCI name of Cocamidopropyl Betaine.

Amphoteric surfactants are understood to be surface-active compounds that include at least one free amino group and at least one —COOH or —SO₃H group in the molecule, in addition to a C₈₋₂₄ alkyl or acyl group, and are capable of forming internal salts. Examples of suitable amphoteric surfactants are N-alkyl glycines, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylaminoacetic acids each having, for instance, 8 to 24 C atoms in the alkyl group. Especially preferred amphoteric surfactants are N-cocoalkylaminopropionate, cocoacylaminoethylaminopropionate, and C₁₂-C₁₈ acylsarco sine.

It has proven advantageous, furthermore, if the dyes and/or lighteners of the invention include further nonionogenic surface-active substances. Nonionic surfactants include as the hydrophilic group, e.g., a polyol group, a polyalkylene glycol ether group, or a combination of polyol and polyglycol ether groups. Such compounds are, for example,
adducts of 1 to 50 mol of ethylene oxide and/or 0 to 5 mol of propylene oxide to linear and branched fatty alcohols having 8 to 30 C atoms, such as, for example, lauryl myristyl, cetyl, but also stearyl, isostearyl, and oleyl alcohol, to fatty acids having 8 to 30 C atoms and to alkyl phenols having 8 to 15 C atoms in the alkyl group,
adducts, end-capped with a methyl or C₂-C₆ alkyl group, of 1 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group such as, for example, the types obtainable under the marketing names Dehydol® LS and Dehydol® LT (Cognis),
polyglycerol esters and alkoxylated polyglycerol esters, such as, for example, poly(3)glycerol diisostearate (commercial product: Lameform® TGI (Henkel)) and poly(2)glycerol polyhydroxystearate (commercial product: Dehymuls® PGPH (Henkel)).
polyol fatty acid esters such as, for example, the commercial product Hydagen® HSP (Cognis) or Sovermol types (Cognis),
higher alkoxylated, preferably propoxylated, and in particular ethoxylated mono-, di-, and triglycerides such as, for example, glycerol monolaurate+20 ethylene oxide and glycerol monostearate+20 ethylene oxide,
amine oxides,
hydroxy mixed ethers,
sorbitan fatty acid esters and adducts of ethylene oxide to sorbitan fatty acid esters, such as, for example, polysorbates, and sorbitan monolaurate+20 mol of ethylene oxide (EO),
sugar fatty acid esters and adducts of ethylene oxide to sugar fatty acid esters,
adducts of ethylene oxide to fatty acid alkanolamides and fatty amines,
fatty acid-N-alkylglucamides,
alkylphenols and alkylphenol alkoxylates having 6 to 21, in particular 6 to 15 carbon atoms in the alkyl chain, and 1 to 30 ethylene oxide and/or propylene oxide units. Preferred representatives of this class are, for example, nonylphenol+9 EO and octylphenol+8 EO;
alkyl polyglycosides corresponding to the general formula RO—(Z), where R stands for alkyl, Z for sugar, and x for the number of sugar units. The alkyl polyglycosides usable according to the invention may include only one specific alkyl group R. Usually, however, these compounds are prepared on the basis of natural fats and oils or mineral oils. In this case, mixtures corresponding to the starting compounds, or corresponding to the respective processing of these compounds, are present as alkyl groups R.

C₈-C₂₂ alkyl mono- and -oligoglycosides and ethoxylated analogs thereof are particularly suitable as nonionic surfactants. The non-ethoxylated compounds in particular have proven to be particularly suitable.

Particularly preferred are alkyl polyglycosides of the formula RO—(Z)ₓ, in which R consists
substantially of C₈ and C₁₀ alkyl groups,
substantially of C₁₂ and C₁₀ alkyl groups,
substantially of C₈ to C₁₆ alkyl groups, or
substantially of C₁₂ to C₁₆ alkyl groups, or
substantially of C₁₆ to C₁₈ alkyl groups.

These compounds are characterized in that any mono- or oligosaccharides may be used as sugar component Z. Sugars having 5 or 6 carbon atoms, as well as the corresponding oligosaccharides, are usually used. Such sugars are, for example, glucose, fructose, galactose, arabinose, ribose, xylose, lyxose, allose, altrose, mannose, gulose, idose, talose, and sucrose. Preferred sugar components are glucose, fructose, galactose, arabinose, and sucrose; glucose is particularly preferred.

The alkyl polyglycosides usable according to the invention include on average 1.1 to 5 sugar units. Alkyl polyglycosides having x values from 1.1 to 2.0 are preferred. Alkyl glycosides in which x is 1.1 to 1.8 are very particularly preferred.

The alkoxylated homologs of the aforesaid alkyl polyglycosides may also be used according to the invention. These homologs may include on average up to 10 ethylene oxide and/or propylene oxide units per alkyl glycoside unit.

Alkylene oxide adducts to saturated, linear fatty alcohols and fatty acids, each with 2 to 30 mol of ethylene oxide per mole of fatty alcohol or fatty acid, have proven to be further preferred nonionic surfactants. Preparations with excellent properties are likewise obtained if they include fatty acid esters of ethoxylated glycerol as the nonionic surfactants.

Particularly preferred nonionogenic surface-active substances here are, on account of the simple processability, substances commercially available in pure form as solids or liquids. In this connection, the definition of purity does not refer to chemically pure compounds. Instead, particularly if they are natural-based products, mixtures of different homologs can be used, for example, with various alkyl chain lengths, as are obtained for products based on natural fats and oils. Also in the case of alkoxylated products, usually mixtures of different degrees of alkoxylation are present. In this connection, the term purity refers rather to the fact that the selected substances should preferably be free from solvents, adjusting agents, and other accompanying substances.

Surfactants which are adducts of ethylene and/or propylene oxide to fatty alcohols or derivatives of these adducts may be used both as products with a “normal” homolog distribution and as products with a narrow homolog distribution. A “normal” homolog distribution is understood to be mixtures of homologs obtained by reacting fatty alcohol and alkylene oxide using alkali metals, alkali metal hydrdoxides, or alkali metal alcoholates as catalysts. Narrow homolog distributions, in contrast, are obtained if, for example, hydrotalcite, alkaline earth metal salts of ether carboxylic acids, alkaline earth metal oxides, hydroxides or alcoholates are used as catalysts. The use of products with a narrow homolog distribution may be preferred.

The anionic, nonionic, zwitterionic, or amphoteric surfactants are used in amounts of 0.1 to 45% by weight, preferably 1 to 30% by weight, and very particularly preferably of 1 to 15% by weight, based on the total amount of the ready-to-use agent.

Cationic surfactants of the quaternary ammonium compound, esterquat, and amidoamine type are also preferred according to the invention in compositions (a) and (b). Preferred quaternary ammonium compounds are ammonium halides, in particular chlorides and bromides, such as alkyltrimethylammonium chlorides, dialkyldimethylammonium chlorides, and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, and the imidazolium compounds known under the INCI names Quaternium-27 and Quaternium-83. The long alkyl chains of the aforesaid surfactants preferably have 10 to 18 carbon atoms. Other cationic surfactants that can be used according to the invention are quaternized protein hydrolysates.

The alkylamidoamines are usually prepared by amidation of natural or synthetic fatty acids and fatty acid cuts with dialkylaminoamines and, apart from a good conditioning effect, are notable especially for their good biodegradability. Stearamidopropyl dimethylamine, commercially available under the name Tegoamid® S 18, represents a compound, especially suitable according to the invention, from this substance group.

Also very highly biodegradable are quaternary ester compounds, the so-called “esterquats.” Esterquats are known substances, which include both at least one ester function and at least one quaternary ammonium group as a structural element. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. The products Armocare® VGH-70, an N,N-bis(2-palmitoyloxyethyl)dimethylammonium chloride, and Dehyquart® F-75, Dehyquart® C-4046, Dehyquart® L80, and Dehyquart® AU-35 are examples of such esterquats.

The cationic surfactants are present in the agents to be used according to the invention preferably in amounts of 0.05 to 10% by weight, based on the total agent. Amounts of 0.1 to 5% by weight are particularly preferred.

In a preferred embodiment, nonionic, zwitterionic, and/or amphoteric surfactants and mixtures thereof may be preferred.

In a further preferred embodiment, the action of the agent of the invention can be enhanced by emulsifiers. Such emulsifiers are, for example,
adducts of 4 to 30 mol of ethylene oxide and/or 0 to 5 mol of propylene oxide to linear fatty alcohols having 8 to 22 C atoms, to fatty acids having 12 to 22 C atoms, and to alkyl phenols having 8 to 15 C atoms in the alkyl group,
C₁₂-C₂₂ fatty acid mono- and diesters of adducts of 1 to 30 mol of ethylene oxide to polyols having 3 to 6 carbon atoms, in particular to glycerol,
ethylene oxide and polyglycerol adducts to methylglucoside fatty acid esters, fatty acid alkanolamides, and fatty acid glucamides,
C₈-C₂₂ alkyl mono- and oligoglycosides and ethoxylated analogs thereof, whereby degrees of oligomerization of 1.1 to 5, in particular 1.2 to 2.0, and glucose as the sugar component are preferred,
mixtures of alkyl (oligo)glucosides and fatty alcohols, for example, the commercially available product Montanov® 68,
adducts of 5 to 60 mol of ethylene oxide to castor oil and hydrogenated castor oil,
partial esters of polyols having 3 to 6 carbon atoms with saturated fatty acids having 8 to 22 C atoms,
sterols, whereby a group of steroids that have a hydroxyl group at C atom 3 of the steroid skeleton, are understood to be sterols and are isolated from both animal tissue (zoosterols) and vegetable fats (phytosterols). Examples of zoosterols are cholesterol and lanosterol. Examples of suitable phytosterols are ergosterol, stigmasterol, and sitosterol. Sterols, the so-called mycosterols, are also isolated from fungi and yeasts.
phospholipids, primarily glucose phospholipids, which are obtained, e.g., as lecithins or phosphatidyl choline from, e.g., egg yolk or plant seeds (e.g., soybeans),
fatty acid esters of sugar and sugar alcohols, such as sorbitol,
polyglycerols and polyglycerol derivatives such as, for example, polyglycerol poly-12-hydroxystearate (commercial product Dehymuls® PGPH),
linear and branched fatty acids having 8 to 30 C atoms and the Na, K, ammonium, Ca, Mg, and Zn salts thereof.

The agents of the invention include the emulsifiers preferably in amounts of 0.1 to 25% by weight, in particular 0.5 to 15% by weight, based on the total amount of the ready-to-use agent.

Nonionogenic emulsifiers or surfactants with an HLB value of 10-15 can be particularly preferred according to the invention. Of the aforesaid emulsifier types, the emulsifiers that include no ethylene oxide and/or propylene oxide in the molecule may be very particularly preferred.

Further, the agents of the invention may include other active substances, auxiliary substances, and additives such as, for example,
nonionic polymers such as vinylpyrrolidinone/vinyl acrylate copolymers, polyvinylpyrrolidinone, vinylpyrrolidinone/vinyl acetate copolymers, polyethylene glycols, and polysiloxanes,
silicones such as volatile or nonvolatile, straight-chain, branched, or cyclic, crosslinked or non-crosslinked polyalkyl siloxanes (such as dimethicones or cyclomethicones), polyaryl siloxanes and/or polyalkylaryl siloxanes, in particular polysiloxanes having organofunctional groups, such as substituted or unsubstituted amines (amodimethicones), carboxyl, alkoxy, and/or hydroxyl groups (dimethicone copolyols), linear polysiloxane(A)-polyoxyalkylene(B) block copolymers, grafted silicone polymers having a non-silicone-containing organic backbone or having a polysiloxane backbone, such as, for example, the commercial product Abil B 8832 of the company Degussa marketed under the INCI name Bis-PEG/PPG-20/20 Dimethicone, or mixtures thereof,
cationic polymers such as quaternized cellulose ethers, polysiloxanes with quaternary groups, dimethyldiallylammonium chloride polymers, acrylamide/dimethyldiallylammonium chloride copolymers, dimethylaminoethylmethacrylate/vinylpyrrolidinone copolymers quaternized with diethylsulfate, vinylpyrrolidinone/imidazolinium methochloride copolymers, and quaternized polyvinyl alcohol,
zwitterionic and amphoteric polymers such as, for example, acrylamidopropyl-trimethylammonium chloride/acrylate copolymers and octylacrylamide/methyl methacrylate/tert-butylaminoethyl methacrylate/2-hydroxypropyl methacrylate copolymers, diallyldimethylammonium chloride/acrylate copolymers, t-butylaminoethyl methacrylate/N-(1, 1,3,3-tetramethylbutyl)acrylamide/acrylate (/methacrylate) copolymers,
anionic polymers such as, for example, polyacrylic acids, crosslinked polyacrylic acids, vinyl acetate/crotonic acid copolymers, vinylpyrrolidinone/vinyl acrylate copolymers, vinyl acetate/butyl maleate/isobornyl acrylate copolymers, methyl vinyl ether/maleic anhydride copolymers, and acrylic acid/ethyl acrylate/N-t-butylacrylamide terpolymers,
other thickeners, such as agar-agar, guar gum, alginates, xanthan gum, gum arabic, karaya gum, locust bean flour, linseed gums, dextrans, cellulose derivatives, e.g., methyl cellulose, hydroxyalkyl cellulose, and carboxymethyl cellulose, starch fractions and derivatives, such as amylose, amylopectin, and dextrins, clays such as, e.g., bentonite or fully synthetic hydrocolloids such as, e.g., polyvinyl alcohol,
structurants such as glucose, maleic acid, and lactic acid,
hair-conditioning compounds such as phospholipids, for example, soya lecithin, egg lecithin, and kephalins, and silicone oils,
perfume oils, dimethyl isosorbide, and cyclodextrins,
solvents and solubilizers such as ethanol, isopropanol, ethylene glycol, propylene glycol, glycerol, and diethylene glycol,
fiber-structure-improving active substances, particularly mono-, di-, and oligosaccharides such as, for example, glucose, galactose, fructose, fruit sugar, and lactose,
quaternized amines such as methyl-1-alkylamidoethyl-2-alkylimidazolinium methosulfate,
defoamers such as silicones,
dyes for coloring the agent,
antidandruff agents such as piroctone olamine, zinc omadine, and climbazole,
amino acids and oligopeptides, in particular arginine and/or serine,
animal- and/or plant-based protein hydrolysates, for example, elastin, collagen, keratin, silk, and milk protein hydrolysates, or almond, rice, pea, potato, and wheat protein hydrolysates, as well as derivatives in the form of fatty acid condensation products thereof or optionally anionically or cationically modified derivatives,
vegetable oils, for example macadamia nut oil, kukui nut oil, palm oil, amaranth seed oil, peach kernel oil, avocado oil, olive oil, coconut oil, rapeseed oil, sesame oil, jojoba oil, soy oil, peanut oil, evening primrose oil, and tea tree oil,
light-protection agents, in particular derivatized benzophenones, cinnamic acid derivatives, and triazines,
substances for adjusting pH, for example, usual acids, in particular edible acids, and bases,
active substances such as panthenol, pantothenic acid, pantolactone, allantoin, pyrrolidinone carboxylic acids, and salts thereof, as well as bisabolol,
polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols,
ceramides, preferably sphingolipids such as ceramide I, ceramide II, ceramide 1, ceramide 2, ceramide 3, ceramide 5, and ceramide 6, or pseudoceramides, such as especially N—(C₈-C₂₂ acyl)-(C₈-C₂₂ acyl)hydroxyproline,
vitamins, provitamins, and vitamin precursors, in particular those of groups A, B₃, B₅, B₆, C, E, F, and H,
plant extracts such as, for example, the extracts from aloe vera, angelica, aniseed, apricot, benzoin, bergamot, birch, stinging nettle, calamus, blackcurrant, costus, marsh mallow, oak bark, elemi, tarragon, spruce needles, galbanum, geranium, ginseng, grapefruit, guaiac wood, green tea, witch hazel, restharrow, hops, coltsfoot, ginger root, iris, jasmine, chamomile, cardamom, clover, burdock root, pine, kiwi, coconut, coriander, caraway, mugo pine, lavender, lemongrass, lily, lime, lime blossom, litchi, mace, mallow, almond, mango, melissa, melon, meristem, myrrh, neroli, olibanum, opoponax, orange, patchouli, petitgrain, stone pine, wild thyme, rooibos, rose, rosemary, horse chestnut, sandalwood, sage, horsetail, yarrow, celery, fir, thyme, juniper, vine leaves, hawthorn, wheat, cuckoo flower, ylang-ylang, cedar, and lemon,
cholesterol,
consistency promoters, such as sugar esters, polyol esters, or polyol alkyl ethers,
fats and waxes such as spermaceti, beeswax, montan wax, and paraffins,
fatty acid alkanolamides,
swelling and penetration agents such as glycerol, propylene glycol monoethyl ether, carbonates, hydrogen carbonates, guanidines, ureas, and primary, secondary, and tertiary phosphates,
opacifiers such as latex, styrene/PVP and styrene/acrylamide copolymers,
pearlescent agents such as ethylene glycol mono- and distearate and PEG-3 distearate,
pigments,
stabilizing agents for hydrogen peroxide and other oxidizing agents,
propellants such as propane-butane mixtures, N₂O, dimethyl ether, CO₂, and air,
antioxidants.

The selection of these additional substances is made by the skilled artisan according to the desired properties of the agents.

Coloring and lightening processes on keratin fibers typically take place in an alkaline environment. To treat keratin fibers and the skin as well as gently as possible, setting a too high pH is not desirable, however. It is preferred, therefore, if the pH of the ready-to-use agent is between 6 and 11, in particular between 7 and 10.5, pH values within the meaning of the present invention are pH values measured at a temperature of 22° C.

Organic alkalinizing agents that can be used according to the invention are preferably selected from alkanolamines of primary, secondary, or tertiary amines with a C₂-C₆ alkyl parent structure, bearing at least one hydroxyl group. Particularly preferred alkanolamines are selected from the group comprising 2-aminoethan-1-ol (monoethanolamine), 2-amino-2-methylpropan-1-ol, 2-amino-2-methylpropane-1,3-diol, and triethanolamine. It has emerged in the context of studies for the present invention, however, that, furthermore, agents preferred according to the invention are characterized in that they include in addition an inorganic alkalinizing agent. The inorganic alkalinizing agent of the invention is preferably selected from the group formed by sodium hydroxide, potassium hydroxide, calcium hydroxide, barium hydroxide, sodium phosphate, potassium phosphate, sodium silicate, potassium silicate, sodium carbonate, and potassium carbonate. Very especially preferred are sodium hydroxide and/or potassium hydroxide. The basic amino acids usable as alkalizing agents of the invention are preferably selected from the group formed by L-arginine, D-arginine, D,L-arginine, L-lysine, D-lysine, D,L-lysine, particularly preferably L-arginine, D-arginine, D,L-arginine used as an alkalizing agent in the context of the invention. Lastly, a further preferred alkalizing agent is ammonia.

With regard to further optional components, as well as the amounts of these components used, reference is made expressly to the relevant manuals known to the skilled artisan, e.g., Kh. Schräder, Grundlagen and Rezepturen der Kosmetika [Cosmetics Fundamentals and Formulations], 2^{nd} ed., Hüthig Buch Verlag, Heidelberg, 1989.

The additional active and auxiliary substances are used in the agents of the invention preferably in each case in amounts of 0.0001 to 10% by weight, in particular of 0.0005 to 5% by weight, based on the total weight of the application mixture.

A further subject of the invention is the use of an agent of the invention to reduce the color shift of dyes on keratinic fibers and/or to improve the wash fastness of dyed keratinic fibers, whereby a good conditioning effect is achieved simultaneously.

Particularly preferred is the use of an agent of the invention to improve the color absorption of the dyes on keratinic fibers and/or to improve the wash fastness of the dyed keratinic fibers.

Particularly suitable for using the agents of the invention is a method for coloring and/or lightening keratinic fibers, in particular human hair, which is characterized in that an agent of the first subject of the invention is applied to the keratin-containing fibers, left on the fibers for 5 to 60 minutes, and then rinsed out again with water or washed out with a shampoo. The contact time of the ready-to-use coloring agents is preferably 5 to 45 minutes, in particular 10 to 40 minutes, particularly preferably 15 to 35 minutes. During the contact time of the agent on the fibers, it may be advantageous to assist the lightening process by supplying heat. Heat may be supplied by an external heat source such as, e.g., warm air from a warm air blower, and also, in particular in the case of hair lightening on living subjects, by the body temperature of the subject. In the case of the latter option, the portion to be colored and/or lightened is usually covered with a hood. A contact phase at room temperature is likewise in accordance with the invention. The temperature during the contact time is in particular between 20° C. and 40° C., in particular between 25° C. and 38° C. Once the contact time has ended, the remaining dye preparation is rinsed out of the hair with water or a cleaning agent. Commercial shampoo, in particular, may serve here as a cleaning agent, whereby the cleaning agent can be omitted and the rinsing-out operation can occur with water, if the lightening agent possesses a high surfactant carrier.

The agents of the present invention may be formulated and correspondingly utilized as three-component agents or as multi-component agents. Separation into multi-component systems is advisable in particular when incompatibilities of the ingredients are a possibility or a risk; in the case of such systems, the agent to be used is prepared by the consumer immediately before use by mixing the components. In the case of an oxidizing coloring method, a coloring and lightening method in which the lightening cream and the oxidizing agent are initially present separately is preferred.

A further subject of the present invention therefore is a method for conditioning and coloring and/or lightening keratinic fibers, which is characterized in that:
if desired, a pretreatment agent M1 is applied to the fibers, then
a coloring and/or lightening agent M2 is used on the fibers, a further agent M3 being added, if desired, to the agent M2 before the application,
said agent M2 is rinsed off the fibers after a time from 5-30 minutes,
and after the treatment, an aftertreatment agent M4 is optionally applied to the fibers and is rinsed off again after a contact time of 2-25 minutes,
   whereby the agent M2 is an agent of the invention.

The statements made about the agents of the invention apply mutatis mutandis in regard to other preferred embodiments of the methods and uses of the invention.

The following examples are intended to explain the subject of the present application in greater detail without, however, limiting it in any form.

### Exemplary Embodiments

Commercially available products were used exclusively. The shades 6-0, 6-88, 6-99, and 7-77 from the series Igora Royal were used as color creams. These color creams were mixed with the typical developer Igora Royal Developer, included in the dye products, in the ratio of 1:1. The commercially available conditioner Schauma Color Shine Conditioner or Gliss Kur Ultimate Color Conditioner was added to this mixture of color cream and developer in an amount of 5 or 10% by weight, based on the ready-to-use mixture of color cream and developer.

Hair strands (Euro Natural Hair, white) were measured colorimetrically (Spectralflasch SF 450 colorimeter from Datacolor) without addition of conditioner in the mixture of color cream and developer, and left at room temperature for 30 minutes. The hair strands were then thoroughly rinsed out and dried in a stream of air. After coloring and drying, the hair strands were again measured colorimetrically. In comparison, the ready-to-use dye preparations prepared as previously described and including the conditioning composition were also placed on the hair strands and left there for 30 minutes at room temperature. The hair strands were then thoroughly rinsed out and dried in a stream of air. After coloring and drying, the hair strands were also measured colorimetrically. The color distance (ΔE) between the strands colored with conditioner in the dye and developer mixture and without conditioner in the dye and developer mixture was calculated according to the following formula:

ΔE=\sqrt{(Lv−Ln)²+(av−an)²+(bv−bn)²}

Lv, av, by colorimetric values without conditioner
Ln, an, bn colorimetric values with conditioner

| | Color shade | | | |
|---|---|---|---|---|
| Euro Natural hair, white, not dyed | 6-0 | 6-88 | 6-99 | 7-77 |
| With 5.0% by weight of Schauma Color | 1.11 | 0.87 | 1.02 | 0.83 |
| Shine Conditioner | | | | |
| With 10.0% by weight of Schauma Color | 1.24 | 0.9 | 0.4 | 0.7 |
| Shine Conditioner | | | | |
| With 5.0% by weight of Gliss Kur | 1.07 | 0.21 | 0.97 | 1.43 |
| Ultimate Conditioner | | | | |
| With 10.0% by weight of Gliss Kur | 0.44 | 0.79 | 0.28 | 0.51 |
| Ultimate Conditioner | | | | |

There is typically a great color shift in the coloration when additives are added to improve combability, care, and gloss. As the addition of the conditioner directly to the dye mixture of color cream and developer shows, in this method only an extremely low color shift is obtained. The smaller the DeltaE value, the smaller the color shift. At ΔE values <2, the color shift is only difficult to perceive with the human eye and not at all perceptible at ΔE values <1.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for coloring and/or lightening keratinic fibers, comprising, in each case in a cosmetic carrier,
(a) a composition including dye precursors,
(b) a composition including an oxidizing agent, and
(c) a conditioning composition.

2. The agent according to claim 1, wherein the conditioning composition comprises 0.1 to 10.0% by weight of a quaternary ammonium compound, the quaternary ammonium compound being selected from the group consisting of:
i. esterquats,
ii. quaternary imidazolines of the formula (Tkat2), 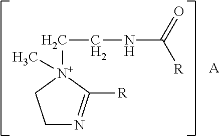
where the R group independently of one another stands in each case for a saturated or unsaturated, linear or branched hydrocarbon group with a chain length of 8 to 30 carbon atoms and A for a physiologically acceptable anion,
iii. the amines and/or cationized amines,
iv. poly(methacryloyloxyethyl)trimethylammonium compounds,
v. quaternized cellulose derivatives, in particular Polyquaternium-10, Polyquaternium-24, Polyquaternium-27, Polyquaternium-67, Polyquaternium-72,
vi. cationic alkyl polyglycosides,
vii. cationized honey,
viii. cationic guar derivatives,
ix. chitosan,
x. polymeric dimethyldiallylammonium salts and copolymers thereof with esters and amides of acrylic acid and methacrylic acid, in particular Polyquaternium-7,
xi. copolymers of vinylpyrrolidone with quaternized derivatives of dialkylaminoalkyl acrylate and methacrylate, in particular Polyquaternium-11,
xii. vinylpyrrolidone/vinylimidazolium methochloride copolymers, in particular Polyquaternium-16,
xiii. quaternized polyvinyl alcohol,
xiv. Polyquaternium-74, and
mixtures thereof.

3. The agent according to claim 1, wherein a mixing ratio of compositions (a) to (b) is 1:1 and the conditioning agent comprises 0.01 to 25 wt % relative to the sum of the total weights of compositions (a) and (b).

4. The agent according to claim 1, wherein a mixing ratio of compositions (a) to (b) is 1:1 and the conditioning agent comprises 0.5 to 23.0% relative to the sum of the total weights of compositions (a) and (b).

5. The agent according to claim 1, wherein a mixing ratio of compositions (a) to (b) is 1:1 and the conditioning agent comprises 0.5 to 15.0% relative to the sum of the total weights of compositions (a) and (b).

6. The agent according to claim 1, wherein a mixing ratio of compositions (a) to (b) is 1:1 and the conditioning agent comprises 4 to 12.0% relative to the sum of the total weights of compositions (a) and (b).

7. The agent according to claim 1, wherein the color-modifying compound includes at least one oxidation dye precursor and/or at least one direct dye.

8. The agent according to claim 7, wherein the at least one oxidation dye precursor comprises 0.001 to 12% by weight of the agent.

9. The agent according to claim 7, wherein the at least one oxidation dye precursor comprises 0.01 to 10% by weight based on the agent.

10. The agent according to claim 7, wherein the at least one oxidation dye precursor comprises 0.1 to 5% by weight of the agent.

11. The agent according to claim 7, wherein the at least one direct dye comprises 0.001 to 12% by weight of the agent.

12. The agent according to claim 1, wherein the color-modifying compound includes at least one oxidizing agent selected from hydrogen peroxide and the solid adducts thereof to organic or inorganic compounds.

13. The agent according to claim 12, wherein the oxidizing agent comprises 0.001 to 12% by weight of the agent.

14. The agent according to claim 12, wherein the oxidizing agent comprises 0.01 to 10% by weight of the agent.

15. The agent according to claim 12, wherein the oxidizing agent comprises 2.5 to 8% by weight of the agent.

16. The agent according to one claim 1, wherein the agent comprises, based on its total weight, 0.01 to 30% of a bleach booster selected from the group consisting of: ammonium peroxodisulfate, potassium peroxodisulfate, sodium peroxodisulfate, potassium hydrogen peroxomonosulfate, potassium peroxodiphosphate, magnesium peroxide, and barium peroxide.

17. A method for coloring and/or lightening keratinic fibers, comprising:
optionally, a pretreatment agent M1 is applied to the fibers, then
a coloring and/or lightening agent M2 is used on the fibers, optionally a further agent M3 being added to the agent M2 before the application,
said agent M2 is rinsed off the fibers after a time from 5-30 minutes,
and after the treatment, an after-treatment agent M4 is optionally applied to the fibers and is rinsed off again after a contact time of 2-25 minutes,
whereby the agent M2 is an agent of the invention according to claim 1.
